(19) United States
(12) Patent Application Publication
(10) Pub. No.: US 2026/0217859 A1
(43) Pub. Date: Jul. 30, 2026

(54) ANTIBODIES TARGETING STEAP-1 AND USES THEREOF

(71) Applicant: Janux Therapeutics, Inc., San Diego, CA (US)

(72) Inventors: David CAMPBELL, San Diego, CA (US); Thomas R. DIRAIMONDO, San Diego, CA (US); John LAUDENSLAGER, San Diego, CA (US)

(21) Appl. No.: 19/639,514

(22) Filed: Apr. 6, 2026

Related U.S. Application Data
(63) Continuation of application No. PCT/US2024/050266, filed on Oct. 7, 2024.
(60) Provisional application No. 63/588,824, filed on Oct. 9, 2023.

(51) Int. Cl. C07K 16/30; C07K 16/28

(52) U.S. Cl. CPC C07K 16/3069; C07K 16/2818; C07K 2317/52; C07K 2317/55; C07K 2317/565; C07K 2317/622; C07K 2317/624

(57) ABSTRACT

Disclosed herein are polypeptides or polypeptide complexes comprising an antibody or antigen binding fragment thereof that binds to six-transmembrane epithelial antigen of the prostate-1 (STEAP-1).

### CROSS-REFERENCE

The present application is a continuation of International Patent Application No. PCT/US2024/050266, filed Oct. 7, 2024, which claims the benefit of U.S. Provisional Application No. 63/588,824 filed on Oct. 9, 2023 which is incorporated herein by reference in its entirety.

### SEQUENCE LISTING

The instant application contains a Sequence Listing which has been submitted electronically in XML format and is hereby incorporated by reference in its entirety. Said XML copy, created on Sep. 26, 2024 is named 52426-759_301SL.xml and is 104,341 bytes in size.

### SUMMARY

Disclosed herein, in some embodiments, are polypeptides or polypeptide complexes comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 61 and Q3, S9, S12, A13, V15, D17, V19, T22, Q35, K48, A49, S66, P86, F89, T91, and Q106 of SEQ ID NO: 62.

In some embodiments, the at least one amino acid mutation is selected from V24A, Y27F, W35M, G45C, T58I, L64V, S66G, T74N, E99D, M111F wherein the amino acid position corresponds to that of SEQ ID NO: 61, and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62. In some embodiments, the at least one amino acid mutation comprises at least two, at least three, at least four, at least five, at least ten, at least 15, or at least 20 mutations selected from V24A, Y27F, W35M, G46C, T58I, L64V, S66G, T74N, E99D, M111F wherein the amino acid position corresponds to that of SEQ ID NO: 61, and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62. In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, M111F wherein the amino acid position corresponds to that of SEQ ID NO: 61, and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62, and the immunoglobulin heavy chain comprises an amino acid sequence of at least 90% sequence identity to SEQ ID NO: 61, and the immunoglobulin light chain comprises an amino acid sequence of at least 90% sequence identity to SEQ ID NO: 62. In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, M111F wherein the amino acid position corresponds to that of SEQ ID NO: 61, and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62, and the immunoglobulin heavy chain comprises an amino acid sequence of at least 95% sequence identity to SEQ ID NO: 61, and the immunoglobulin light chain comprises an amino acid sequence of at least 95% sequence identity to SEQ ID NO: 62.

In some embodiments, the immunoglobulin heavy chain comprises mutations V24A, Y27F, T58I, and T74N, and at least 90% sequence identity to SEQ ID NO: 61. In some embodiments, the immunoglobulin heavy chain comprises mutations W35M and L64V and at least 90% sequence identity to SEQ ID NO: 61. In some embodiments, the immunoglobulin heavy chain comprises mutations V24A, Y27F, W35M, T58I, L64V, S66G, and T74N and at least 90% sequence identity to SEQ ID NO: 61. In some embodiments, the immunoglobulin heavy chain comprises mutations W35M, L64V, E99D, and M111F and at least 90% sequence identity to SEQ ID NO: 61. In some embodiments, the immunoglobulin heavy chain comprises mutations V24A, Y27F, W35M, L64V, T74N, E99D, and M111F and at least 90% sequence identity to SEQ ID NO: 61. In some embodiments, the immunoglobulin heavy chain comprises the G45C mutation and at least 90% sequence identity to SEQ ID NO: 61. In some embodiments, the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 62. In some embodiments, the immunoglobulin light chain comprises the Q35N mutation and at least 90% sequence identity to SEQ ID NO: 62. In some embodiments, the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62. In some embodiments, the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62. In some embodiments, the immunoglobulin light chain comprises the Q106C mutation and at least 90% sequence identity to SEQ ID NO: 62.

In some embodiments, the immunoglobulin heavy chain comprises mutations V24A, Y27F, T58I, and T74N, and at least 90% sequence identity to SEQ ID NO: 61, and the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 62. In some embodiments, the immunoglobulin heavy chain comprises mutations W35M and L64V and at least 90% sequence identity to SEQ ID NO: 61, and the immunoglobulin light chain comprises the Q35N mutation and at least 90% sequence identity to SEQ ID NO: 62. In some embodiments, the immunoglobulin heavy chain comprises mutations V24A, Y27F, W35M, T58I, L64V, S66G, and T74N and at least 90% sequence identity to SEQ ID NO: 61, and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62. In some embodiments, the immunoglobulin heavy chain comprises mutations W35M, L64V, E99D, and M111F and at least 90% sequence identity to SEQ ID NO: 61, and the immunoglobulin light chain comprises the Q35N mutation and at least 90% sequence identity to SEQ ID NO: 62. In some embodiments, the immunoglobulin heavy chain comprises mutations V24A, Y27F, W35M, L64V, T74N, E99D, and M111F and at least 90% sequence identity to SEQ ID NO: 61, and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62. In some embodiments, the immunoglobulin heavy chain comprises the G45C mutation and at least 90% sequence identity to SEQ ID NO: 61 and the immunoglobulin light chain comprises the Q106C mutation and at least 90% sequence identity to SEQ ID NO: 62. In some embodiments, the antibody or antigen binding fragment thereof comprises an engineered disulfide bond between the immunoglobulin heavy chain and the immunoglobulin light chain. In some embodiments, the antibody or antigen binding fragment thereof comprises an engineered disulfide bond between the cysteine at the position of the G45C mutation and the cysteine at the position of the Q106C mutation.

In some embodiments, the immunoglobulin light chain comprises complementarity determining regions (CDRs) CDR1-L, CDR2-L, and CDR3-L, and the immunoglobulin heavy chain comprises CDRs: CDR1-H, CDR2-H, and CDR3-H, wherein at least one of the CDRs comprises the amino acid sequence selected from SEQ ID NOs: 3-38. In some embodiments, CDR1-H comprises the amino acid sequence selected from SEQ ID NOs: 3-8, CDR2-H comprises the amino acid sequence selected from SEQ ID NOs: 9-14, and CDR3-H comprises the amino acid sequence selected from SEQ ID NOs: 15-20. In some embodiments, CDR1-L comprises the amino acid sequence selected from SEQ ID NOs: 21-26, CDR2-L comprises the amino acid sequence selected from SEQ ID NOs: 27-32, and CDR3-L comprises the amino acid sequence selected from SEQ ID NOs: 33-38. In some embodiments, CDR1-H comprises the amino acid sequence selected from SEQ ID NOs: 3-8, CDR2-H comprises the amino acid sequence selected from SEQ ID NOs: 9-14, CDR3-H comprises the amino acid sequence selected from SEQ ID NOs: 15-20, CDR1-L comprises the amino acid sequence selected from SEQ ID NOs: 21-26, CDR2-L comprises the amino acid sequence selected from SEQ ID NOs: 27-32, and CDR3-L comprises the amino acid sequence selected from SEQ ID NOs: 33-38.

In some embodiments, the immunoglobulin heavy chain comprises the G45C mutation and the immunoglobulin light chain comprises the Q106C mutation, and wherein the polypeptide or polypeptide complex comprises an amino acid sequence having at least 95%, 98%, 99%, or 100% sequence identity to SEQ ID NO: 89. In some embodiments, the immunoglobulin heavy chain comprises the G45C mutation and the immunoglobulin light chain comprises the Q106C mutation, and wherein the polypeptide or polypeptide complex comprises an amino acid sequence having at least 95%, 98%, 99%, or 100% sequence identity to SEQ ID NO: 90.

Disclosed herein, in some embodiments, are polypeptides or polypeptide complexes comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

In some embodiments, the immunoglobulin heavy chain and the immunoglobulin light chain comprise a set of six CDR sequences selected from the group consisting of: CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33; CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

In some embodiments, the immunoglobulin light chain comprises a variable domain of an immunoglobulin kappa (IgK) or immunoglobulin lambda (IgL) light chain. In some embodiments, the immunoglobulin heavy chain comprises a constant domain derived from an IgG1, IgG2, IgG3, or IgG4 heavy chain.

In some embodiments, the immunoglobulin light chain and the immunoglobulin heavy chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34, and the immunoglobulin heavy chain comprises an amino acid sequence of at least 95% sequence identity to SEQ ID NO: 69 and the immunoglobulin light chain comprises an amino acid sequence of at least 95% sequence identity to SEQ ID NO: 70. In some embodiments, the immunoglobulin heavy chain comprises an amino acid sequence of at least 99% sequence identity to SEQ ID NO: 69 and the immunoglobulin light chain comprises an amino acid sequence of at least 99% sequence identity to SEQ ID NO: 70. In some embodiments, the immunoglobulin heavy chain comprises the amino acid sequence of SEQ ID NO: 69 and the immunoglobulin light chain comprises the amino acid sequence of SEQ ID NO: 70. In some embodiments, the immunoglobulin light chain and the heavy chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; and wherein the immunoglobulin heavy chain comprises an amino acid sequence of at least 95% sequence identity to SEQ ID NO: 39 and the immunoglobulin light chain comprises an amino acid sequence of at least 95% sequence identity to SEQ ID NO: 40. In some embodiments, the immunoglobulin heavy chain comprises an amino acid sequence of at least 99% sequence identity to SEQ ID NO: 39 and the immunoglobulin light chain comprises an amino acid sequence of at least 99% sequence identity to SEQ ID NO: 40. In some embodiments, the immunoglobulin heavy chain comprises the amino acid sequence of SEQ ID NO: 39 and the immunoglobulin light chain comprises the amino acid sequence of SEQ ID NO: 40.

In some embodiments, the immunoglobulin light chain and the immunoglobulin heavy chain comprise a set of six CDR sequences of CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; and wherein the immunoglobulin heavy chain comprises an amino acid sequence of at least 95% sequence identity to SEQ ID NO: 71 and the immunoglobulin light chain comprises an amino acid sequence of at least 95% sequence to SEQ ID NO: 72. In some embodiments, the immunoglobulin heavy chain comprises an amino acid sequence of at least 99% sequence identity to SEQ ID NO: 71 and the immunoglobulin light chain comprises an amino acid sequence of at least 99% sequence identity to SEQ ID NO: 72. In some embodiments, the immunoglobulin heavy chain comprises the amino acid sequence of SEQ ID NO: 71 and the immunoglobulin light chain comprises the amino acid sequence of SEQ ID NO: 72.

In some embodiments, the immunoglobulin light chain and the immunoglobulin heavy chain comprise a set of six CDR sequences of CDR1-H: SEQ ID NO: 5, CDR2-H: SEQ ID NO: 11, CDR3-H: SEQ ID NO: 17, CDR1-L SEQ ID NO: 23, CDR2-L: SEQ ID NO: 29, CDR3-L: SEQ ID NO: 35; and wherein the immunoglobulin heavy chain comprises an amino acid sequence of at least 95% sequence identity to SEQ ID NO: 41 and the immunoglobulin light chain comprises an amino acid sequence of at least 95% sequence identity to SEQ ID NO: 42. In some embodiments, the immunoglobulin heavy chain comprises an amino acid sequence of at least 99% sequence identity to SEQ ID NO: 41 and the immunoglobulin light chain comprises an amino acid sequence of at least 99% sequence identity to SEQ ID NO: 42. In some embodiments, the immunoglobulin heavy chain comprises the amino acid sequence of SEQ ID NO: 41 and the immunoglobulin light chain comprises the amino acid sequence of SEQ ID NO: 42.

In some embodiments, the immunoglobulin light chain and the immunoglobulin heavy chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; and wherein the immunoglobulin heavy chain comprises an amino acid sequence of at least 95% sequence identity to SEQ ID NO: 73 and the immunoglobulin light chain comprises an amino acid sequence of at least 95% sequence to SEQ ID NO: 74. In some embodiments, the immunoglobulin heavy chain comprises an amino acid sequence of at least 99% sequence identity to SEQ ID NO: 73 and the immunoglobulin light chain comprises an amino acid sequence of at least 99% sequence identity to SEQ ID NO: 74. In some embodiments, the immunoglobulin heavy chain comprises the amino acid sequence of SEQ ID NO: 73 and the immunoglobulin light chain comprises the amino acid sequence of SEQ ID NO: 74. In some embodiments, the immunoglobulin light chain and the immunoglobulin heavy chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36, and the immunoglobulin heavy chain comprises an amino acid sequence of at least 95% sequence identity to SEQ ID NO: 43 and the immunoglobulin light chain comprises an amino acid sequence of at least 95% sequence identity to SEQ ID NO: 44. In some embodiments, the immunoglobulin heavy chain comprises an amino acid sequence of at least 99% sequence identity to SEQ ID NO: 43 and the immunoglobulin light chain comprises an amino acid sequence of at least 99% sequence identity to SEQ ID NO: 44. In some embodiments, the immunoglobulin heavy chain comprises the amino acid sequence of SEQ ID NO: 43 and the immunoglobulin light chain comprises the amino acid sequence of SEQ ID NO: 44.

In some embodiments, the immunoglobulin light chain and the immunoglobulin heavy chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; and wherein the immunoglobulin heavy chain comprises an amino acid sequence of at least 95% sequence identity to SEQ ID NO: 75 and the immunoglobulin light chain comprises an amino acid sequence of at least 95% sequence to SEQ ID NO: 76. In some embodiments, the immunoglobulin heavy chain comprises an amino acid sequence of at least 99% sequence identity to SEQ ID NO: 75 and the immunoglobulin light chain comprises an amino acid sequence of at least 99% sequence identity to SEQ ID NO: 76. In some embodiments, the immunoglobulin heavy chain comprises the amino acid sequence of SEQ ID NO: 75 and the immunoglobulin light chain comprises the amino acid sequence of SEQ ID NO: 76. In some embodiments, the immunoglobulin light chain and the immunoglobulin heavy chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37, and the immunoglobulin heavy chain comprises an amino acid sequence of at least 95% sequence identity to SEQ ID NO: 45 and the immunoglobulin light chain comprises an amino acid sequence of at least 95% sequence identity to SEQ ID NO: 46. In some embodiments, the immunoglobulin heavy chain comprises an amino acid sequence of at least 99% sequence identity to SEQ ID NO: 45 and the immunoglobulin light chain comprises an amino acid sequence of at least 99% sequence identity to SEQ ID NO: 46. In some embodiments, the immunoglobulin heavy chain comprises the amino acid sequence of SEQ ID NO: 45 and the immunoglobulin light chain comprises the amino acid sequence of SEQ ID NO: 46.

In some embodiments, the immunoglobulin light chain and the immunoglobulin heavy chain comprise a set of six CDR sequences of CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38; and wherein the immunoglobulin heavy chain comprises an amino acid sequence of at least 95% sequence identity to SEQ ID NO: 77 and the immunoglobulin light chain comprises an amino acid sequence of at least 95% sequence to SEQ ID NO: 78. In some embodiments, the immunoglobulin heavy chain comprises an amino acid sequence of at least 99% sequence identity to SEQ ID NO: 77 and the immunoglobulin light chain comprises an amino acid sequence of at least 99% sequence identity to SEQ ID NO: 78. In some embodiments, the immunoglobulin heavy chain comprises the amino acid sequence of SEQ ID NO: 77 and the immunoglobulin light chain comprises the amino acid sequence of SEQ ID NO: 78.

In some embodiments, the immunoglobulin light chain and the immunoglobulin heavy chain comprise a set of six CDR sequences of CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38, and the immunoglobulin heavy chain comprises an amino acid sequence of at least 95% sequence identity to SEQ ID NO: 47 and the immunoglobulin light chain comprises an amino acid sequence of at least 95% sequence identity to SEQ ID NO: 48. In some embodiments, the immunoglobulin heavy chain comprises an amino acid sequence of at least 99% sequence identity to SEQ ID NO: 47 and the immunoglobulin light chain comprises an amino acid sequence of at least 99% sequence identity to SEQ ID NO: 48. In some embodiments, the immunoglobulin heavy chain comprises the amino acid sequence of SEQ ID NO: 47 and the immunoglobulin light chain comprises the amino acid sequence of SEQ ID NO: 48.

In some embodiments, the immunoglobulin heavy chain comprises an amino acid sequence of at least 90%, 95%, 98%, 99%, or 100% sequence identity to SEQ ID NO: 79 and the immunoglobulin light chain comprises an amino acid sequence of at least 90%, 95%, 98%, 99%, or 100% sequence identity to SEQ ID NO: 80. In some embodiments, the immunoglobulin heavy chain comprises the amino acid sequence of SEQ ID NO: 79 and the immunoglobulin light chain comprises the amino acid sequence of SEQ ID NO: 80. In some embodiments, the antibody or antigen binding fragment thereof comprises an amino acid sequence of at least 90%, 95%, 98%, 99%, or 100% sequence identity to SEQ ID NO: 89. In some embodiments, the antibody or antigen binding fragment thereof comprises the amino acid sequence of SEQ ID NO: 89. In some embodiments, the antibody or the antigen binding fragment thereof comprises an amino acid sequence of at least 90%, 95%, 98%, 99%, or 100% sequence identity to SEQ ID NO: 90. In some embodiments, the antibody or the antigen binding fragment thereof comprises the amino acid sequence of SEQ ID NO: 90.

In some embodiments, the immunoglobulin heavy chain comprises amino acid mutations K154E and K220E, wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 90% sequence identity to SEQ ID NO: 83 and the immunoglobulin light chain comprises an amino acid sequence of at least 90% sequence identity to SEQ ID NO: 84. In some embodiments, the immunoglobulin heavy chain comprises amino acid mutations K154E and K220E, wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 95% sequence identity to SEQ ID NO: 83 and the immunoglobulin light chain comprises an amino acid sequence of at least 95% sequence identity to SEQ ID NO: 84.

In some embodiments, the immunoglobulin heavy chain comprises an amino acid sequence of at least 99% sequence identity to SEQ ID NO: 49 and the immunoglobulin light chain comprises an amino acid sequence of at least 99% sequence identity to SEQ ID NO: 50. In some embodiments, the immunoglobulin heavy chain comprises an amino acid sequence of at least 99% sequence identity to SEQ ID NO: 51 and the immunoglobulin light chain comprises an amino acid sequence of at least 99% sequence identity to SEQ ID NO: 52. In some embodiments, the immunoglobulin heavy chain comprises the amino acid sequence of SEQ ID NO: 51 and the immunoglobulin light chain comprises the amino acid sequence of SEQ ID NO: 52. In some embodiments, the immunoglobulin heavy chain comprises an amino acid sequence of at least 99% sequence identity to SEQ ID NO: 53 and the immunoglobulin light chain comprises an amino acid sequence of at least 99% sequence identity to SEQ ID NO: 54. In some embodiments, the immunoglobulin heavy chain comprises the amino acid sequence of SEQ ID NO: 53 and the immunoglobulin light chain comprises the amino acid sequence of SEQ ID NO: 54. In some embodiments, the immunoglobulin heavy chain comprises an amino acid sequence of at least 99% sequence identity to SEQ ID NO: 55 and the immunoglobulin light chain comprises an amino acid sequence of at least 99% sequence identity to SEQ ID NO: 56. In some embodiments, the immunoglobulin heavy chain comprises the amino acid sequence of SEQ ID NO: 55 and the immunoglobulin light chain comprises the amino acid sequence of SEQ ID NO: 56. In some embodiments, the immunoglobulin heavy chain comprises an amino acid sequence of at least 99% sequence identity to SEQ ID NO: 57 and the immunoglobulin light chain comprises an amino acid sequence of at least 99% sequence identity to SEQ ID NO: 58. In some embodiments, the immunoglobulin heavy chain comprises the amino acid sequence of SEQ ID NO: 57 and the immunoglobulin light chain comprises the amino acid sequence of SEQ ID NO: 58. In some embodiments, the immunoglobulin heavy chain comprises an amino acid sequence of at least 99% sequence identity to SEQ ID NO: 59 and the immunoglobulin light chain comprises an amino acid sequence of at least 99% sequence identity to SEQ ID NO: 60. In some embodiments, the immunoglobulin heavy chain comprises the amino acid sequence of SEQ ID NO: 59 and the immunoglobulin light chain comprises the amino acid sequence of SEQ ID NO: 60.

In some embodiments, the polypeptide or polypeptide complex that comprises the antibody or antigen binding fragment thereof that binds to STEAP-1 is a monoclonal antibody. In some embodiments, the polypeptide or polypeptide complex that comprises the antibody or antigen binding fragment thereof that binds to STEAP-1 is a monospecific antibody, bispecific antibody, or trispecific antibody. In some embodiments, the antibody or antigen binding fragment thereof that binds to STEAP-1 comprises a Fab, Fab′, (Fab′)₂ or a single chain variable fragment (scFv). In some embodiments, the antibody or antigen binding fragment thereof that binds to STEAP-1 comprises the Fab. In some embodiments, the antibody or antigen binding fragment thereof that binds to STEAP-1 comprises the scFv. In some embodiments, a C-terminus of the immunoglobulin light chain of the scFv is connected to an N-terminus of the immunoglobulin heavy chain of the scFv via a linker (L). In some embodiments, a C-terminus of the immunoglobulin heavy chain of the scFv is connected to an N-terminus of the immunoglobulin light chain of the scFv via a linker (L).

In some embodiments, the polypeptide or polypeptide complex comprises more than one antibody or antigen binding fragment thereof that binds to STEAP-1. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L1). In some embodiments, L₁ connects an immunoglobulin heavy chain of the Fab or Fab′ that binds STEAP-1 with another immunoglobulin heavy chain of the second Fab or Fab′ that binds STEAP-1. In some embodiments, L₁ connects an immunoglobulin light chain of the Fab or Fab′ that binds STEAP-1 with another immunoglobulin light chain of the second Fab or Fab′ that binds STEAP-1. In some embodiments, L₁ connects an immunoglobulin light chain of the Fab or Fab′ that binds STEAP-1 with an immunoglobulin heavy chain of the second Fab or Fab′ that binds STEAP-1. In some embodiments, L or L₁ comprises an amino acid sequence selected from any one of SEQ ID NOs: 91-96.

In some embodiments, the polypeptide or polypeptide complex comprises an antibody or antigen binding fragment thereof that binds to a second antigen other than STEAP-1.

Disclosed herein, in some embodiments, are pharmaceutical compositions comprising: (i) the polypeptide or polypeptide complex of any one of the embodiments disclosed herein; and (ii) a pharmaceutically acceptable excipient.

Disclosed herein, in some embodiments, are pharmaceutical compositions comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 61 and Q3, S9, S12, A13, V15, D17, V19, T22, Q35, K48, A49, S66, P86, F89, T91, and Q106 of SEQ ID NO: 62.

Disclosed herein, in some embodiments, are pharmaceutical compositions comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

Disclosed herein, in some embodiments, are isolated recombinant nucleic acid molecules encoding a polypeptide of the antibody or antigen binding fragment thereof of any one of the embodiments disclosed herein.

Disclosed herein, in some embodiments, are isolated recombinant nucleic acid molecules encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 61 and Q3, S9, S12, A13, V15, D17, V19, T22, Q35, K48, A49, S66, P86, F89, T91, and Q106 of SEQ ID NO: 62.

Disclosed herein, in some embodiments, are isolated recombinant nucleic acid molecules encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

Disclosed herein, in some embodiments, are vectors comprising a nucleic acid molecule encoding a polypeptide of the antibody or antigen binding fragment thereof of any of the embodiments disclosed herein.

Disclosed herein, in some embodiments, are host cells comprising a polypeptide of the antibody or antigen binding fragment thereof of any of the embodiments disclosed herein.

Disclosed herein are methods of treating cancer in a subject in need thereof comprising administering to the subject the polypeptide or polypeptide complex of any of the embodiments disclosed herein. In some embodiments, the cancer is sarcoma or a cancer of the prostate, breast, pancreas, bladder, gastrointestinal tract, testis, ovary, or cervix. In some embodiments, the cancer is prostate cancer or Ewing sarcoma. In some embodiments, the method further comprises administering to the subject an immune checkpoint inhibitor. In some embodiments, the immune checkpoint inhibitor comprises an antibody or antigen binding fragment thereof that binds to PD-1.

### DETAILED DESCRIPTION OF THE INVENTION

A tumor-associated antigen of therapeutic interest is six-transmembrane epithelial antigen of prostate 1 (STEAP-1). STEAP-1 is a membrane protein that is up-regulated in multiple cancer lines such as prostate, bladder, colon, ovarian, and Ewing sarcoma, among others. Given its increased expression in cancer tissues, STEAP-1 is a promising target for antibody-based cancer therapy. Disclosed herein are various wild-type and mutated polypeptide or polypeptide complexes that bind to STEAP-1. In some embodiments, the polypeptide or polypeptide complexes described herein are used in a method of treating cancer. In some embodiments, the cancer has cells that express or overexpress STEAP-1. In some embodiments, the cancer is sarcoma or a cancer of the prostate, breast, pancreas, bladder, gastrointestinal tract, testis, ovary, or cervix.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like. The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2: 106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

As used herein, the terms “immunoglobulin light chain” and “immunoglobulin heavy chain” include at least the variable domain and may include one or more constant domains.

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc MP et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg NR and Rees AR, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Polypeptide or Polypeptide Complexes that Bind to STEAP-1

Disclosed herein, in some embodiments, are polypeptides or polypeptide complexes comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M11I of SEQ ID NO: 61 and Q3, S9, S12, A13, V15, D17, V19, T22, Q35, K48, A49, S66, P86, F89, T91, and Q106 of SEQ ID NO: 62 (see Tables 1-2).

TABLE 1
Anti-STEAP-1 Template Sequence

| | | SEQ ID |
|---|---|---|
| Description | Amino Acid Sequence | NO: |
| Anti-STEAP-1 | EVQLVESGGGLVQPGGSLRLSCAVSGYSITSDYA | 1 |
| Fab Heavy Chain | WNWVRQAPGKGLEWVGYISNSGSTSYNPSLKSR | |
| (SBD-1/wt) | FTISRDTSKNTLYLQMNSLRAEDTAVYYCARER | |
| | NYDYDDYYYAMDYWGQGTLVTVSSASTKGPSV | |
| | FPLAPSSKSTSGGTAALGCLVKDYFPEPVTVSWN | |
| | SGALTSGVHTFPAVLQSSGLYSLSSVVTVPSSSLG | |
| | TQTYICNVNHKPSNTKVDKKVEPKSC | |
| Anti-STEAP-1 | EVQLVESGGGLVQPGGSLRLSCAVSGYSITSDYA | 61 |
| Heavy Chain | WNWVRQAPGKGLEWVGYISNSGSTSYNPSLKSR | |
| Variable Domain | FTISRDTSKNTLYLQMNSLRAEDTAVYYCARER | |
| (SBD-1/wt) | NYDYDDYYYAMDYWGQGTLVTVSS | |
| Anti-STEAP-1 | DIQMTQSPSSLSASVGDRVTITCKSSQSLLYRSNQ | 2 |
| Fab Light Chain | KNYLAWYQQKPGKAPKLLIYWASTRESGVPSRF | |
| (SBD-1/wt) | SGSGSGTDFTLTISSLQPEDFATYYCQQYYNYPR | |
| | TFGQGTKVEIKRTVAAPSVFIFPPSDEQLKSGTAS | |
| | VVCLLNNFYPREAKVQWKVDNALQSGNSQESV | |
| | TEQDSKDSTYSLSSTLTLSKADYEKHKVYACEVT | |
| | HQGLSSPVTKSFNRGEC | |
| Anti-STEAP-1 | DIQMTQSPSSLSASVGDRVTITCKSSQSLLYRSNQ | 62 |
| Light Chain | KNYLAWYQQKPGKAPKLLIYWASTRESGVPSRF | |
| Variable Domain | SGSGSGTDFTLTISSLQPEDFATYYCQQYYNYPR | |
| (SBD-1/wt) | TFGQGTKVEIK | |

TABLE 2
Anti-STEAP-1 Mutations

| Chain | Position or Mutation |
|---|---|
| Exemplary amino acid positions for mutation - | V24, Y27, G45, W35, T58, L64, S66, T74, E99, |
| Heavy Chain | M111 |
| (numbering corresponding to SEQ ID NO: 61) | |
| Exemplary amino acid positions and mutations - | V24A, Y27F, W35M, G45C, T58I, L64V, S66G, |
| Heavy Chain | T74N, E99D, M111F |
| (numbering corresponding to SEQ ID NO: 61) | |
| Exemplary amino acid positions for mutation - | Q3, S9, S12, A13, V15, D17, V19, T22, Q35, K48, |
| Light Chain | A49, S66, P86, F89, T91, Q106 |
| (numbering corresponding to SEQ ID NO: 62) | |
| Exemplary amino acid positions and mutations - | Q3V, S9D, S12A, A13V, V15L, D17E, V19A, T22N, |
| Light Chain | Q35N, K48Q, A49P, S66D, P86A, F89V, T91V, |
| (numbering corresponding to SEQ ID NO: 62) | Q106C |

In some embodiments, the at least one amino acid mutation is selected from V24A, Y27F, W35M, G45C, T58I, L64V, S66G, T74N, E99D), and M111F wherein the amino acid position corresponds to that of SEQ ID NO: 61, and Q3V, S9D), S12A, A13V, V15L, D17E, V19A, T22N, Q35N, K48Q, A49P, S66D, P86A, F89V, T91V, and Q1106C wherein the amino acid position corresponds to that of SEQ ID NO: 62. In some embodiments, the at least one amino acid mutation is V24A wherein the amino acid position corresponds to that of SEQ ID NO: 61. In some embodiments, the at least one amino acid mutation is Y27F wherein the amino acid position corresponds to that of SEQ ID NO: 61. In some embodiments, the at least one amino acid mutation is W35M wherein the amino acid position corresponds to that of SEQ ID NO: 61. In some embodiments, the at least one amino acid mutation is G45C wherein the amino acid position corresponds to that of SEQ ID NO: 61. In some embodiments, the at least one amino acid mutation is T58I wherein the amino acid position corresponds to that of SEQ ID NO: 61. In some embodiments, the at least one amino acid mutation is L64V wherein the amino acid position corresponds to that of SEQ ID NO: 61. In some embodiments, the at least one amino acid mutation is S66G wherein the amino acid position corresponds to that of SEQ ID NO: 61. In some embodiments, the at least one amino acid mutation is T74N wherein the amino acid position corresponds to that of SEQ ID NO: 61. In some embodiments, the at least one amino acid mutation E99D wherein the amino acid position corresponds to that of SEQ ID NO: 61. In some embodiments, the at least one amino acid mutation is M111F wherein the amino acid position corresponds to that of SEQ ID NO: 61. In some embodiments, the at least one amino acid mutation is Q3V wherein the amino acid position corresponds to that of SEQ ID NO: 62. In some embodiments, the at least one amino acid mutation is S9D wherein the amino acid position corresponds to that of SEQ ID NO: 62. In some embodiments, the at least one amino acid mutation is S12A wherein the amino acid position corresponds to that of SEQ ID NO: 62. In some embodiments, the at least one amino acid mutation is A13V wherein the amino acid position corresponds to that of SEQ ID NO: 62. In some embodiments, the at least one amino acid mutation is V15L wherein the amino acid position corresponds to that of SEQ ID NO: 62. In some embodiments, the at least one amino acid mutation is D17E wherein the amino acid position corresponds to that of SEQ ID NO: 62. In some embodiments, the at least one amino acid mutation is V19A wherein the amino acid position corresponds to that of SEQ ID NO: 62. In some embodiments, the at least one amino acid mutation is T22N wherein the amino acid position corresponds to that of SEQ ID NO: 62. In some embodiments, the at least one amino acid mutation is Q35N wherein the amino acid position corresponds to that of SEQ ID NO: 62. In some embodiments, the at least one amino acid mutation is K48Q wherein the amino acid position corresponds to that of SEQ ID NO: 62. In some embodiments, the at least one amino acid mutation is A49P wherein the amino acid position corresponds to that of SEQ ID NO: 62. In some embodiments, the at least one amino acid mutation is S66D wherein the amino acid position corresponds to that of SEQ ID NO: 62. In some embodiments, the at least one amino acid mutation is P86A wherein the amino acid position corresponds to that of SEQ ID NO: 62. In some embodiments, the at least one amino acid mutation is F89V wherein the amino acid position corresponds to that of SEQ ID NO: 62. In some embodiments, the at least one amino acid mutation is T91V wherein the amino acid position corresponds to that of SEQ ID NO: 62. In some embodiments, the at least one amino acid mutation is Q106C wherein the amino acid position corresponds to that of SEQ ID NO: 62.

In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61, and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62. In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61. In some embodiments, the at least one amino acid mutation comprises at least two, at least three, at least four, at least five, at least ten, at least 15, or at least 20 mutations selected from Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62.

In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62, and the immunoglobulin heavy chain comprises an amino acid sequence with at least 90% sequence identity to SEQ ID NO: 61, and the immunoglobulin light chain comprises an amino acid sequence with at least 90% sequence identity to SEQ ID NO: 62. In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61 and the immunoglobulin heavy chain comprises an amino acid sequence with at least 90% sequence identity to SEQ ID NO: 61. In some embodiments, the at least one amino acid mutation comprises at least two, at least three, at least four, at least five, at least ten, at least 15, or at least 20 mutations selected from Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62 and the immunoglobulin light chain comprises an amino acid sequence with at least 90% sequence identity to SEQ ID NO: 62.

In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62, and the immunoglobulin heavy chain comprises an amino acid sequence with at least 92% sequence identity to SEQ ID NO: 61, and the immunoglobulin light chain comprises an amino acid sequence with at least 92% sequence identity to SEQ ID NO: 62. In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62, and the immunoglobulin heavy chain comprises an amino acid sequence with at least 95% sequence identity to SEQ ID NO: 61, and the immunoglobulin light chain comprises an amino acid sequence with at least 95% sequence identity to SEQ ID NO: 62. In some embodiments, the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61 and the immunoglobulin heavy chain comprises an amino acid sequence with at least 95% sequence identity to SEQ ID NO: 61. In some embodiments, the at least one amino acid mutation comprises at least two, at least three, at least four, at least five, at least ten, at least 15, or at least 20 mutations selected from Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62 and the immunoglobulin light chain comprises an amino acid sequence with at least 95% sequence identity to SEQ ID NO: 62.

In some embodiments, the immunoglobulin heavy chain comprises mutations V24A, Y27F, T58I, and T74N, and at least 90% sequence identity to SEQ ID NO: 61. In some embodiments, the immunoglobulin heavy chain comprises mutations V24A, Y27F, T58I, and T74N, and at least 92% sequence identity to SEQ ID NO: 61. In some embodiments, the immunoglobulin heavy chain comprises mutations V24A, Y27F, T58I, and T74N, and at least 95% sequence identity to SEQ ID NO: 61. In some embodiments, the immunoglobulin heavy chain comprises mutations V24A, Y27F, T58I, and T74N, and at least 98% sequence identity to SEQ ID NO: 61.

In some embodiments, the immunoglobulin heavy chain comprises mutations W35M and L64V and at least 90% sequence identity to SEQ ID NO: 61. In some embodiments, the immunoglobulin heavy chain comprises mutations W35M and L64V and at least 92% sequence identity to SEQ ID NO: 61. In some embodiments, the immunoglobulin heavy chain comprises mutations W35M and L64V and at least 95% sequence identity to SEQ ID NO: 61. In some embodiments, the immunoglobulin heavy chain comprises mutations W35M and L64V and at least 98% sequence identity to SEQ ID NO: 61.

In some embodiments, the immunoglobulin heavy chain comprises mutations V24A, Y27F, W35M, T58I, L64V, S66G, and T74N and at least 90% sequence identity to SEQ ID NO: 61. In some embodiments, the immunoglobulin heavy chain comprises mutations V24A, Y27F, W35M, T58I, L64V, S66G, and T74N and at least 92% sequence identity to SEQ ID NO: 51. In some embodiments, the immunoglobulin heavy chain comprises mutations V24A, Y27F, W35M, T58I, L64V, S66G, and T74N and at least 95% sequence identity to SEQ ID NO: 61.

In some embodiments, the immunoglobulin heavy chain comprises mutations W35M, L64V, E99D, and M111F and at least 90% sequence identity to SEQ ID NO: 61. In some embodiments, the immunoglobulin heavy chain comprises mutations W35M, L64V, E99D, and M111F and at least 92% sequence identity to SEQ ID NO: 61. In some embodiments, the immunoglobulin heavy chain comprises mutations W35M, L64V, E99D, and M111F and at least 95% sequence identity to SEQ ID NO: 61.

In some embodiments, the immunoglobulin heavy chain comprises mutations V24A, Y27F, W35M, L64V, T74N, E99D, and M111F and at least 90% sequence identity to SEQ ID NO: 61. In some embodiments, the immunoglobulin heavy chain comprises mutations V24A, Y27F, W35M, L64V, T74N, E99D, and M111F and at least 92% sequence identity to SEQ ID NO: 61. In some embodiments, the immunoglobulin heavy chain comprises mutations V24A, Y27F, W35M, L64V, T74N, E99D, and M111F and at least 95% sequence identity to SEQ ID NO: 61.

In some embodiments, the immunoglobulin heavy chain comprises a G45C mutation and at least 90% sequence identity to SEQ ID NO: 61. In some embodiments, the immunoglobulin heavy chain comprises a G45C mutation and at least 92% sequence identity to SEQ ID NO: 61. In some embodiments, the immunoglobulin heavy chain comprises a G45C mutation and at least 95% sequence identity to SEQ ID NO: 61. In some embodiments, the immunoglobulin heavy chain comprises a G45C mutation and at least 98% sequence identity to SEQ ID NO: 61.

In some embodiments, the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 62. In some embodiments, the immunoglobulin light chain comprises mutations Q3V, S9D, S12A, A13V, V15L, D17E, V19A, T22N, K48Q, A49P, S66D, P86A, F89V, and T91V and at least 92% sequence identity to SEQ ID NO: 62. In some embodiments, the immunoglobulin light chain comprises mutations Q3V, S9D, S12A, A13V, V15L, D17E, V19A, T22N, K48Q, A49P, S66D, P86A, F89V, and T91V and at least 95% sequence identity to SEQ ID NO: 62.

In some embodiments, the immunoglobulin light chain comprises the Q35N mutation and at least 90% sequence identity to SEQ ID NO: 62. In some embodiments, the immunoglobulin light chain comprises the Q35N mutation and at least 92% sequence identity to SEQ ID NO: 62. In some embodiments, the immunoglobulin light chain comprises the Q35N mutation and at least 95% sequence identity to SEQ ID NO: 62. In some embodiments, the immunoglobulin light chain comprises the Q35N mutation and at least 99% sequence identity to SEQ ID NO: 62.

In some embodiments, the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62. In some embodiments, the immunoglobulin light chain comprises mutations Q3V, S9D, S12A, A13V, V15L, D17E, V19A, T22N, Q35N, K48Q, A49P, S66D, P86A, F89V, and T91V and at least 92% sequence identity to SEQ ID NO: 62. In some embodiments, the immunoglobulin light chain comprises mutations Q3V, S9D, S12A, A13V, V15L, D17E, V19A, T22N, Q35N, K48Q, A49P, S66D, P86A, F89V, and T91V and at least 95% sequence identity to SEQ ID NO: 62.

In some embodiments, the immunoglobulin light chain comprises the Q106C mutation and at least 90% sequence identity to SEQ ID NO: 62. In some embodiments, the immunoglobulin light chain comprises the Q106C mutation and at least 92% sequence identity to SEQ ID NO: 62. In some embodiments, the immunoglobulin light chain comprises the Q106C mutation and at least 95% sequence identity to SEQ ID NO: 62.

In some embodiments, the immunoglobulin heavy chain comprises mutations V24A, Y27F, T58I, and T74N, and at least 90% sequence identity to SEQ ID NO: 61 and the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 62. In some embodiments, the immunoglobulin heavy chain comprises mutations V24A, Y27F, T58I, and T74N, and at least 92% sequence identity to SEQ ID NO: 61 and the immunoglobulin light chain comprises mutations Q3V, S9D, S12A, A13V, V15L, D17E, V19A, T22N, K48Q, A49P, S66D, P86A, F89V, and T91V and at least 92% sequence identity to SEQ ID NO: 62. In some embodiments, the immunoglobulin heavy chain comprises mutations V24A, Y27F, T58I, and T74N, and at least 95% sequence identity to SEQ ID NO: 61 and the immunoglobulin light chain comprises mutations Q3V, S9D, S12A, A13V, V15L, D17E, V19A, T22N, K48Q, A49P, S66D, P86A, F89V, and T91V and at least 95% sequence identity to SEQ ID NO: 62.

In some embodiments, the immunoglobulin heavy chain comprises mutations W35M and L64V and at least 90% sequence identity to SEQ ID NO: 61 and the immunoglobulin light chain comprises the Q35N mutation and at least 90% sequence identity to SEQ ID NO: 62. In some embodiments, the immunoglobulin heavy chain comprises mutations W35M and L64V and at least 92% sequence identity to SEQ ID NO: 61 and the immunoglobulin light chain comprises the Q35N mutation and at least 92% sequence identity to SEQ ID NO: 62. In some embodiments, the immunoglobulin heavy chain comprises mutations W35M and L64V and at least 95% sequence identity to SEQ ID NO: 61 and the immunoglobulin light chain comprises the Q35N mutation and at least 95% sequence identity to SEQ ID NO: 62. In some embodiments, the immunoglobulin heavy chain comprises mutations W35M and L64V and at least 99% sequence identity to SEQ ID NO: 61, and the immunoglobulin light chain comprises the Q35N mutation and at least 99% sequence identity to SEQ ID NO: 62.

In some embodiments, the immunoglobulin heavy chain comprises mutations V24A, Y27F, W35M, T58I, L64V, S66G, and T74N and at least 90% sequence identity to SEQ ID NO: 61 and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62. In some embodiments, the immunoglobulin heavy chain comprises mutations V24A, Y27F, W35M, T58I, L64V, S66G, and T74N and at least 92% sequence identity to SEQ ID NO: 61 and the immunoglobulin light chain comprises mutations Q3V, S9D, S12A, A13V, V15L, D17E, V19A, T22N, Q35N, K48Q, A49P, S66D, P86A, F89V, and T91V and at least 92% sequence identity to SEQ ID NO: 62. In some embodiments, the immunoglobulin heavy chain comprises mutations V24A, Y27F, W35M, T58I, L64V, S66G, and T74N and at least 95% sequence identity to SEQ ID NO: 61 and the immunoglobulin light chain comprises mutations Q3V, S9D, S12A, A13V, V15L, D17E, V19A, T22N, Q35N, K48Q, A49P, S66D, P86A, F89V, and T91V and at least 95% sequence identity to SEQ ID NO: 62.

In some embodiments, the immunoglobulin heavy chain comprises mutations W35M, L64V, E99D, and M111F and at least 90% sequence identity to SEQ ID NO: 61 and the immunoglobulin light chain comprises the Q35N mutation and at least 90% sequence identity to SEQ ID NO: 62. In some embodiments, the immunoglobulin heavy chain comprises mutations W35M, L64V, E99D, and M111F and at least 92% sequence identity to SEQ ID NO: 61 and the immunoglobulin light chain comprises the Q35N mutation and at least 92% sequence identity to SEQ ID NO: 62. In some embodiments, the immunoglobulin heavy chain comprises mutations W35M, L64V, E99D, and M111F and at least 95% sequence identity to SEQ ID NO: 61 and the immunoglobulin light chain comprises the Q35N mutation and at least 95% sequence identity to SEQ ID NO: 62. In some embodiments, the immunoglobulin heavy chain comprises mutations W35M, L64V, E99D, and M111F and at least 98% sequence identity to SEQ ID NO: 61 and the immunoglobulin light chain comprises the Q35N mutation and at least 98% sequence identity to SEQ ID NO: 62.

In some embodiments, the immunoglobulin heavy chain comprises mutations V24A, Y27F, W35M, L64V, T74N, E99D, and M111F and at least 90% sequence identity to SEQ ID NO: 61 and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62. In some embodiments, the immunoglobulin heavy chain comprises mutations V24A, Y27F, W35M, L64V, T74N, E99D, and M111F and at least 92% sequence identity to SEQ ID NO: 61 and the immunoglobulin light chain comprises mutations Q3V, S9D, S12A, A13V, V15L, D17E, V19A, T22N, Q35N, K48Q, A49P, S66D, P86A, F89V, and T91V and at least 92% sequence identity to SEQ ID NO: 62. In some embodiments, the immunoglobulin heavy chain comprises mutations V24A, Y27F, W35M, L64V, T74N, E99D, and M111F and at least 95% sequence identity to SEQ ID NO: 61 and the immunoglobulin light chain comprises mutations Q3V, S9D, S12A, A13V, V15L, D17E, V19A, T22N, Q35N, K48Q, A49P, S66D, P86A, F89V, and T91V and at least 95% sequence identity to SEQ ID NO: 62.

In some embodiments, the immunoglobulin heavy chain comprises the G45C mutation and at least 90% sequence identity to SEQ ID NO: 61 and the immunoglobulin light chain comprises the Q106C mutation and at least 90% sequence identity to SEQ ID NO: 62. In some embodiments, the immunoglobulin heavy chain comprises the G45C mutation and at least 92% sequence identity to SEQ ID NO: 61 and the immunoglobulin light chain comprises the Q106C mutation and at least 92% sequence identity to SEQ ID NO: 62. In some embodiments, the immunoglobulin heavy chain comprises the G45C mutation and at least 95% sequence identity to SEQ ID NO: 61 and the immunoglobulin light chain comprises the Q106C mutation and at least 95% sequence identity to SEQ ID NO: 62. In some embodiments, the antibody or antigen binding fragment thereof comprises an engineered disulfide bond between the immunoglobulin heavy chain and the immunoglobulin light chain. In some embodiments, the antibody or antigen binding fragment thereof comprises an engineered disulfide bond between the cysteine at the position of the G45C mutation and the cysteine at the position of the Q106C mutation.

In some embodiments, the immunoglobulin light chain comprises complementarity determining regions (CDRs) CDR1-L, CDR2-L, and CDR3-L, and the immunoglobulin heavy chain comprises CDRs: CDR1-H, CDR2-H, and CDR3-H, wherein at least one of the CDRs comprises the amino acid sequence selected from SEQ ID NOs: 3-38. In some embodiments, CDR1-L comprises the amino acid sequence of SEQ ID NO: 23. In some embodiments, CDR1-L comprises the amino acid sequence selected from SEQ ID NOs: 21-26. In some embodiments, CDR1-L comprises the amino acid sequence selected from SEQ ID NOs: 23-26. In some embodiments, CDR2-L comprises the amino acid sequence selected from SEQ ID NOs: 27-32. In some embodiments, CDR3-L comprises the amino acid sequence selected from SEQ ID NOs 33-38 In some embodiments, CDR2-H comprises the amino acid sequence selected from SEQ ID NOs: 3-8. In some embodiments, CDR1-H comprises the amino acid sequence selected from SEQ ID NOs: 5-8. In some embodiments, CDR2-H comprises the amino acid sequence selected from SEQ ID NOs: 9-14. In some embodiments, CDR2-H comprises the amino acid sequence of SEQ ID NO: 10. In some embodiments, CDR2-H comprises the amino acid sequence of SEQ ID NO: 11. In some embodiments, CDR2-H comprises the amino acid sequence of SEQ ID NO: 12. In some embodiments, CDR2-H comprises the amino acid sequence selected from SEQ ID NOs: 13-14. In some embodiments, CDR3-H comprises the amino acid sequence selected from SEQ ID NOs: 15-20. In some embodiments, CDR3-H comprises the amino acid sequence selected from SEQ ID NOs: 19-20.

TABLE 3
Anti-STEAP Heavy Chain CDR
sequences as (according to Kabat)

| Con- | | SEQ | | SEQ | | SEQ |
|---|---|---|---|---|---|---|
| struct | | ID | | ID | | ID |
| Name | CDR1-H | NO: | CDR2-H | NO: | CDR3-H | NO: |
| HC CDR- | SDYAWN | 3 | YISNSGSTS | 9 | ERNYDYDD | 15 |
| wt | | | YNPSLKS | | YYYAMDY | |
| HC CDR- | SDYAWN | 4 | YISNSGSIS | 10 | ERNYDYDD | 16 |
| T58I | | | YNPSLKS | | YYYAMDY | |
| HC CDR- | SDYAMN | 5 | YISNSGSTS | 11 | ERNYDYDD | 17 |
| W35M, | | | YNPSVKS | | YYYAMDY | |
| L64V | | | | | | |
| HC CDR- | SDYAMN | 6 | YISNSGSIS | 12 | ERNYDYDD | 18 |
| W35M, | | | YNPSVKG | | YYYAMDY | |
| T58I, | | | | | | |
| L64V, | | | | | | |
| S66G | | | | | | |
| HC CDR- | SDYAMN | 7 | YISNSGSTS | 13 | DRNYDYDD | 19 |
| W35M, | | | YNPSVKS | | YYYAFDY | |
| L64V, | | | | | | |
| E99D, | | | | | | |
| M111F | | | | | | |
| HC CDR- | SDYAMN | 8 | YISNSGSTS | 14 | DRNYDYDD | 20 |
| W35M, | | | YNPSVKS | | YYYAFDY | |
| L64V, | | | | | | |
| E99D, | | | | | | |
| M111F | | | | | | |
| HC CDR | TYWIE | 63 | EILPGSGQT | 64 | WGYYGTRG | 65 |
| | | | DFNEKFQG | | YFNV | |

TABLE 4
Anti-STEAP Light Chain CDR
sequences as (according to Kabat)

| Con- | | SEQ | | SEQ | | SEQ |
|---|---|---|---|---|---|---|
| struct | | ID | | ID | | ID |
| Name | CDR1-L | NO: | CDR2-L | NO: | CDR3-L | NO: |
| LC CDR- | KSSQSLLYR | 21 | WASTRES | 27 | QQYYNYPRT | 33 |
| wt | SNQKNYLA | | | | | |
| LC CDR- | KSSQSLLYR | 22 | WASTRES | 28 | QQYYNYPRT | 34 |
| wt | SNQKNYLA | | | | | |
| LC CDR- | KSSQSLLYR | 23 | WASTRES | 29 | QQYYNYPRT | 35 |
| Q35N | SNNKNYLA | | | | | |
| LC CDR- | KSSQSLLYR | 24 | WASTRES | 30 | QQYYNYPRT | 36 |
| Q35N | SNNKNYLA | | | | | |
| LC CDR- | KSSQSLLYR | 25 | WASTRES | 31 | QQYYNYPRT | 37 |
| Q35N | SNNKNYLA | | | | | |
| LC CDR- | KSSQSLLYR | 26 | WASTRES | 32 | QQYYNYPRT | 38 |
| Q35N | SNNKNYLA | | | | | |
| LC CDR | RASSSVSYM | 66 | STSNLAS | 67 | QQRRSFPYT | 68 |
| | H | | | | | |

In some embodiments, CDR1-H comprises the amino acid sequence selected from SEQ ID NO s: 3-8, CDR2-H comprises the amino acid sequence selected from SEQ ID NOs: 9-14, and CDR3-H comprises the amino acid sequence selected from SEQ ID NOs: 15-20. In some embodiments, CDR1-L comprises the amino acid sequence selected from SEQ ID NOs: 21-26, CDR2-L comprises the amino acid sequence selected from SEQ ID NOs: 27-32, and CDR3-L comprises the amino acid sequence selected from SEQ ID NOs: 33-38. In some embodiments, CDR1-H comprises the amino acid sequence selected from SEQ ID NOs: 3-8, CDR2-H comprises the amino acid sequence selected from SEQ ID NOs: 9-14, CDR3-H comprises the amino acid sequence selected from SEQ ID NOs: 15-20, CDR1-L comprises the amino acid sequence selected from SEQ ID NOs: 21-26, CDR2-L comprises the amino acid sequence selected from SEQ ID NOs: 27-32, and CDR3-L comprises the amino acid sequence selected from SEQ ID NOs: 33-38.

In some embodiments, the immunoglobulin heavy chain comprises the G45C mutation and the immunoglobulin light chain comprises the Q106C mutation, and wherein the polypeptide or polypeptide complex comprises an amino acid sequence having at least 95%, 98%, 99%, or 10000 sequence identity to SEQ ID NO: 89. In some embodiments, the immunoglobulin heavy chain comprises the G45C mutation and the immunoglobulin light chain comprises the Q106C mutation, and wherein the polypeptide or polypeptide complex comprises an amino acid sequence having at least 95% sequence identity to SEQ ID NO: 89. In some embodiments, the immunoglobulin heavy chain comprises the G45C mutation and the immunoglobulin light chain comprises the Q106C mutation, and wherein the polypeptide or polypeptide complex comprises an amino acid sequence having at least 98% sequence identity to SEQ ID NO: 89. In some embodiments, the immunoglobulin heavy chain comprises the G45C mutation and the immunoglobulin light chain comprises the Q106C mutation, and wherein the polypeptide or polypeptide complex comprises an amino acid sequence having at least 99% sequence identity to SEQ ID NO: 89. In some embodiments, the immunoglobulin heavy chain comprises the G45C mutation and the immunoglobulin light chain comprises the Q106C mutation, and wherein the polypeptide or polypeptide complex comprises an amino acid sequence having at least 100% sequence identity to SEQ ID NO: 89.

In some embodiments, the immunoglobulin heavy chain comprises the G45C mutation and the immunoglobulin light chain comprises the Q106C mutation, and wherein the polypeptide or polypeptide complex comprises an amino acid sequence having at least 95%, 98%, 99%, or 100% sequence identity to SEQ ID NO: 90. In some embodiments, the immunoglobulin heavy chain comprises the G45C mutation and the immunoglobulin light chain comprises the Q106C mutation, and wherein the polypeptide or polypeptide complex comprises an amino acid sequence having at least 95% sequence identity to SEQ ID NO: 90. In some embodiments, the immunoglobulin heavy chain comprises the G45C mutation and the immunoglobulin light chain comprises the Q106C mutation, and wherein the polypeptide or polypeptide complex comprises an amino acid sequence having at least 98% sequence identity to SEQ ID NO: 90. In some embodiments, the immunoglobulin heavy chain comprises the G45C mutation and the immunoglobulin light chain comprises the Q106C mutation, and wherein the polypeptide or polypeptide complex comprises an amino acid sequence having at least 99% sequence identity to SEQ ID NO: 90. In some embodiments, the immunoglobulin heavy chain comprises the G45C mutation and the immunoglobulin light chain comprises the Q106C mutation, and wherein the polypeptide or polypeptide complex comprises an amino acid sequence having at least 90% sequence identity to SEQ ID NO: 89.

Disclosed herein, in some embodiments, are polypeptides or polypeptide complexes comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

In some embodiments, the immunoglobulin heavy chain and the immunoglobulin light chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34. In some embodiments, the immunoglobulin heavy chain and the immunoglobulin light chain comprise a set of six CDR sequences of CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35. In some embodiments, the immunoglobulin heavy chain and the immunoglobulin light chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36. In some embodiments, the immunoglobulin heavy chain and the immunoglobulin light chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37. In some embodiments, the immunoglobulin heavy chain and the immunoglobulin light chain comprise a set of six CDR sequences of CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

In some embodiments, the immunoglobulin heavy chain and the immunoglobulin light chain comprise a set of six CDR sequences selected from the group consisting of: CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, and CDR3-L: SEQ ID NO: 33; CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

In some embodiments, the immunoglobulin light chain comprises a variable domain of an immunoglobulin kappa (IgK) or immunoglobulin lambda (IgL) light chain. In some embodiments, the immunoglobulin heavy chain comprises a constant domain derived from an IgG1, IgG2, IgG3, or IgG4 heavy chain.

In some embodiments, the immunoglobulin light chain and the immunoglobulin heavy chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34, and the immunoglobulin heavy chain comprises an amino acid sequence of at least 95% sequence identity to SEQ ID NO: 69 and the immunoglobulin light chain comprises an amino acid sequence of at least 95% sequence identity to SEQ ID NO: 70. In some embodiments, the immunoglobulin heavy chain comprises an amino acid sequence of at least 99% sequence identity to SEQ ID NO: 69 and the immunoglobulin light chain comprises an amino acid sequence of at least 99% sequence identity to SEQ ID NO: 70. In some embodiments, the immunoglobulin heavy chain comprises the amino acid sequence of SEQ ID NO: 69 and the immunoglobulin light chain comprises the amino acid sequence of SEQ ID NO: 70.

In some embodiments, the immunoglobulin light chain and the immunoglobulin heavy chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34, and the immunoglobulin heavy chain comprises an amino acid sequence of at least 95% sequence identity to SEQ ID NO: 39 and the immunoglobulin light chain comprises an amino acid sequence of at least 95% sequence identity to SEQ ID NO: 40. In some embodiments, the immunoglobulin heavy chain comprises an amino acid sequence of at least 99% sequence identity to SEQ ID NO: 39 and the immunoglobulin light chain comprises an amino acid sequence of at least 99% sequence identity to SEQ ID NO: 40. In some embodiments, the immunoglobulin heavy chain comprises the amino acid sequence of SEQ ID NO: 39 and the immunoglobulin light chain comprises the amino acid sequence of SEQ ID NO: 40.

In some embodiments, the immunoglobulin light chain and the immunoglobulin heavy chain comprise a set of six CDR sequences of CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35, and the immunoglobulin heavy chain comprises an amino acid sequence of at least 95% sequence identity to SEQ ID NO: 71 and the immunoglobulin light chain comprises an amino acid sequence of at least 95% sequence identity to SEQ ID NO: 72. In some embodiments, the immunoglobulin heavy chain comprises an amino acid sequence of at least 99% sequence identity to SEQ ID NO: 71 and the immunoglobulin light chain comprises an amino acid sequence of at least 99% sequence identity to SEQ ID NO: 72. In some embodiments, the immunoglobulin heavy chain comprises the amino acid sequence of SEQ ID NO: 71 and the immunoglobulin light chain comprises the amino acid sequence of SEQ ID NO: 72.

In some embodiments, the immunoglobulin light chain and the immunoglobulin heavy chain comprise a set of six CDR sequences of CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35, and the immunoglobulin heavy chain comprises an amino acid sequence of at least 95% sequence identity to SEQ ID NO: 41 and the immunoglobulin light chain comprises an amino acid sequence of at least 95% sequence identity to SEQ ID NO: 42. In some embodiments, the immunoglobulin heavy chain comprises an amino acid sequence of at least 99% sequence identity to SEQ ID NO: 41 and the immunoglobulin light chain comprises an amino acid sequence of at least 99% sequence identity to SEQ ID NO: 42. In some embodiments, the immunoglobulin heavy chain comprises the amino acid sequence of SEQ ID NO: 41 and the immunoglobulin light chain comprises the amino acid sequence of SEQ ID NO: 42.

In some embodiments, the immunoglobulin light chain and the immunoglobulin heavy chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36, and the immunoglobulin heavy chain comprises an amino acid sequence of at least 95% sequence identity to SEQ ID NO: 73 and the immunoglobulin light chain comprises an amino acid sequence of at least 95% sequence identity to SEQ ID NO: 74. In some embodiments, the immunoglobulin heavy chain comprises an amino acid sequence of at least 99% sequence identity to SEQ ID NO: 73 and the immunoglobulin light chain comprises an amino acid sequence of at least 99% sequence identity to SEQ ID NO: 74. In some embodiments, the immunoglobulin heavy chain comprises the amino acid sequence of SEQ ID NO: 73 and the immunoglobulin light chain comprises the amino acid sequence of SEQ ID NO: 74.

In some embodiments, the immunoglobulin light chain and the immunoglobulin heavy chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36, and the immunoglobulin heavy chain comprises an amino acid sequence of at least 95% sequence identity to SEQ ID NO: 43 and the immunoglobulin light chain comprises an amino acid sequence of at least 95% sequence identity to SEQ ID NO: 44. In some embodiments, the immunoglobulin heavy chain comprises an amino acid sequence of at least 99% sequence identity to SEQ ID NO: 43 and the immunoglobulin light chain comprises an amino acid sequence of at least 99% sequence identity to SEQ ID NO: 44. In some embodiments, the immunoglobulin heavy chain comprises the amino acid sequence of SEQ ID NO: 43 and the immunoglobulin light chain comprises the amino acid sequence of SEQ ID NO: 44.

In some embodiments, the immunoglobulin light chain and the immunoglobulin heavy chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37, and the immunoglobulin heavy chain comprises an amino acid sequence of at least 95% sequence identity to SEQ ID NO: 75 and the immunoglobulin light chain comprises an amino acid sequence of at least 95% sequence identity to SEQ ID NO: 76. In some embodiments, the immunoglobulin heavy chain comprises an amino acid sequence of at least 99% sequence identity to SEQ ID NO: 75 and the immunoglobulin light chain comprises an amino acid sequence of at least 99% sequence identity to SEQ ID NO: 76. In some embodiments, the immunoglobulin heavy chain comprises the amino acid sequence of SEQ ID NO:75 and the immunoglobulin light chain comprises the amino acid sequence of SEQ ID NO: 76.

In some embodiments, the immunoglobulin light chain and the immunoglobulin heavy chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37, and the immunoglobulin heavy chain comprises an amino acid sequence of at least 95% sequence identity to SEQ ID NO: 45 and the immunoglobulin light chain comprises an amino acid sequence of at least 95% sequence identity to SEQ ID NO: 46. In some embodiments, the immunoglobulin heavy chain comprises an amino acid sequence of at least 99% sequence identity to SEQ ID NO: 45 and the immunoglobulin light chain comprises an amino acid sequence of at least 99% sequence identity to SEQ ID NO: 46. In some embodiments, the immunoglobulin heavy chain comprises the amino acid sequence of SEQ ID NO: 45 and the immunoglobulin light chain comprises the amino acid sequence of SEQ ID NO: 46.

In some embodiments, the immunoglobulin light chain and the immunoglobulin heavy chain comprise a set of six CDR sequences of CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38, and the immunoglobulin heavy chain comprises an amino acid sequence of at least 95% sequence identity to SEQ ID NO: 77 and the immunoglobulin light chain comprises an amino acid sequence of at least 95% sequence identity to SEQ ID NO: 78. In some embodiments, the immunoglobulin heavy chain comprises an amino acid sequence of at least 99% sequence identity to SEQ ID NO: 77 and the immunoglobulin light chain comprises an amino acid sequence of at least 99% sequence identity to SEQ ID NO: 78. In some embodiments, the immunoglobulin heavy chain comprises the amino acid sequence of SEQ ID NO: 77 and the immunoglobulin light chain comprises the amino acid sequence of SEQ ID NO: 78.

In some embodiments, the immunoglobulin light chain and the immunoglobulin heavy chain comprise a set of six CDR sequences of CDR-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38, and the immunoglobulin heavy chain comprises an amino acid sequence of at least 9500 sequence identity to SEQ ID NO: 47 and the immunoglobulin light chain comprises an amino acid sequence of at least 95% sequence identity to SEQ ID NO: 48. In some embodiments, the immunoglobulin heavy chain comprises an amino acid sequence of at least 99% sequence identity to SEQ ID NO: 47 and the immunoglobulin light chain comprises an amino acid sequence of at least 99% sequence identity to SEQ ID NO: 48. In some embodiments, the immunoglobulin heavy chain comprises the amino acid sequence of SEQ ID NO: 47 and the immunoglobulin light chain comprises the amino acid sequence of SEQ ID NO: 48.

TABLE 5
Anti-STEAP-1 Variable Heavy Chain
and Variable Light Chain Sequences

| Construct | | SEQ | | SEQ |
|---|---|---|---|---|
| Name/ | | ID | | ID |
| mutations | Heavy Chain | NO: | Light Chain | NO: |
| SBD-2/HC: | EVQLVESGGGLVQPGGSLRLSCAAS | 69 | DIVMTQSPDSLAVSLGERA | 70 |
| V24A, Y27F, | GFSITSDYAWNWVRQAPGKGLEWV | | TINCKSSQSLLYRSNQKNYL | |
| T58I, T74N | GYISNSGSISYNPSLKSRFTISRDNSK | | AWYQQKPGQPPKLLIYWAS | |
| LC: Q3V, | NTLYLQMNSLRAEDTAVYYCARER | | TRESGVPDRFSGSGSGTDFT | |
| S9D, S12A, | NYDYDDYYYAMDYWGQGTLVTVSS | | LTISSLQAEDVAVYYCQQY | |
| A13V, V15L, | | | YNYPRTFGQGTKVEIK | |
| D17E, V19A, | | | | |
| T22N, K48Q, | | | | |
| A49P, S66D, | | | | |
| P86A, F89V, | | | | |
| T91V | | | | |
| SBD-3/HC: | EVQLVESGGGLVQPGGSLRLSCAVS | 71 | DIQMTQSPSSLSASVGDRVT | 72 |
| W35M, L64V | GYSITSDYAMNWVRQAPGKGLEW | | ITCKSSQSLLYRSNNKNYLA | |
| LC: Q35N | VGYISNSGSTSYNPSVKSRFTISRDT | | WYQQKPGKAPKLLIYWAS | |
| | SKNTLYLQMNSLRAEDTAVYYCAR | | TRESGVPSRFSGSGSGTDFT | |
| | ERNYDYDDYYYAMDYWGQGTLVT | | LTISSLQPEDFATYYCQQYY | |
| | VSS | | NYPRTFGQGTKVEIK | |
| SBD-4/HC: | EVQLVESGGGLVQPGGSLRLSCAAS | 73 | DIVMTQSPDSLAVSLGERA | 74 |
| V24A, Y27F, | GFSITSDYAMNWVRQAPGKGLEWV | | TINCKSSQSLLYRSNNKNYL | |
| W35M, T58I, | GYISNSGSISYNPSVKGRFTISRDNS | | AWYQQKPGQPPKLLIYWAS | |
| L64V, S66G, | KNTLYLQMNSLRAEDTAVYYCARE | | TRESGVPDRFSGSGSGTDFT | |
| T74N | RNYDYDDYYYAMDYWGQGTLVT | | LTISSLQAEDVAVYYCQQY | |
| LC: Q3V, | VSS | | YNYPRTFGQGTKVEIK | |
| S9D, S12A, | | | | |
| A13V, V15L, | | | | |
| D17E, V19A, | | | | |
| T22N, Q35N, | | | | |
| K48Q, A49P, | | | | |
| S66D, P86A, | | | | |
| F89V, T91V | | | | |
| SBD-5/HC: | EVQLVESGGGLVQPGGSLRLSCAVS | 75 | DIQMTQSPSSLSASVGDRVT | 76 |
| W35M, L64V, | GYSITSDYAMNWVRQAPGKGLEW | | ITCKSSQSLLYRSNNKNYLA | |
| E99D, M111F | VGYISNSGSTSYNPSVKSRFTISRDT | | WYQQKPGKAPKLLIYWAS | |
| LC: Q35N | SKNTLYLQMNSLRAEDTAVYYCAR | | TRESGVPSRFSGSGSGTDFT | |
| | DRNYDYDDYYYAFDYWGQGTLVT | | LTISSLQPEDFATYYCQQYY | |
| | VSS | | NYPRTFGQGTKVEIK | |
| SBD-6/HC: | EVQLVESGGGLVQPGGSLRLSCAAS | 77 | DIVMTQSPDSLAVSLGERA | 78 |
| V24A, Y27F, | GFSITSDYAMNWVRQAPGKGLEWV | | TINCKSSQSLLYRSNNKNYL | |
| W35M, L64V, | GYISNSGSTSYNPSVKSRFTISRDNS | | AWYQQKPGQPPKLLIYWAS | |
| T74N, E99D, | KNTLYLQMNSLRAEDTAVYYCARD | | TRESGVPDRFSGSGSGTDFT | |
| M111F | RNYDYDDYYYAFDYWGQGTLVTV | | LTISSLQAEDVAVYYCQQY | |
| LC: Q3V, | SS | | YNYPRTFGQGTKVEIK | |
| S9D, S12A, | | | | |
| A13V, V15L, | | | | |
| D17E, V19A, | | | | |
| T22N, Q35N, | | | | |
| K48Q, A49P, | | | | |
| S66D, P86A, | | | | |
| F89V, T91V | | | | |
| SBD-7 | EVQLVESGGGLVQPGGSLRLSCAVS | 97 | DIQMTQSPSSLSASVGDRVT | 98 |
| | GYSITSDYAWNWVRQAPGKGLEW | | ITCKSSQSLLYRSNQKNYLA | |
| | VGYISNSGSTSYNPSLKSRFTISRDTS | | WYQQKPGKAPKLLIYWAS | |
| | KNTLYLQMNSLRAEDTAVYYCARE | | TRESGVPSRFSGSGSGTDFT | |
| | RNYDYDDYYYAMDYWGQGTLVT | | LTISSLQPEDFATYYCQQYY | |
| | VSS | | NYPRTFGQGTKVEIK | |
| SBD-8 | QVQLVQSGAEVKKPGASVKVSCKA | 99 | EIVLTQSPATLSLSPGERATL | 100 |
| | SGYTFSTYWIEWVRQAPGQRLEWM | | SCRASSSVSYMHWFQQKPG | |
| | GEILPGSGQTDFNEKFQGRVTFTAD | | QAPRLLIYSTSNLASGIPARF | |
| | TSSDTAYMELSSLRSEDTAVYYCTR | | SGSGSGTDYTLTISSLEPEDF | |
| | WGYYGTRGYFNVWGQGTLVTVSS | | AVYYCQQRRSFPYTFGQGT | |
| | | | KLEIK | |
| SBD-9 | EVQLVESGGGLVQPGGSLRLSCAVS | 101 | DIQMTQSPSSLSASVGDRVT | 102 |
| | GYSITSDYAWNWVRQAPGKGLEW | | ITCKSSQSLLYRSNQKNYLA | |
| | VGYISNSGSTSYNPSLKSRFTISRDTS | | WYQQKPGKAPKLLIYWAS | |
| | KNTLYLQMNSLRAEDTAVYYCARE | | TRESGVPSRFSGSGSGTDFT | |
| | RNYDYDDYYYAMDYWGQGTLVT | | LTISSLQPEDFATYYCQQYY | |
| | VSS | | NYPRTFGQGTKVEIK | |
| SBD-10 | EVQLVESGGGLVQPGGSLRLSCAVS | 103 | DIQMTQSPSSLSASVGDRVT | 104 |
| | GYSITSDYAWNWVRQAPGKGLEW | | ITCKSSQSLLYRSNQKNYLA | |
| | VGYISNSGSTSYNPSLKSRFTISRDTS | | WYQQKPGKAPKLLIYWAS | |
| | KNTLYLQMNSLRAEDTAVYYCARE | | TRESGVPSRFSGSGSGTDFT | |
| | RNYDYDDYYYAMDYWGQGTLVT | | LTISSLQPEDFATYYCQQYY | |
| | VSS | | NYPRTFGQGTKVEIK | |
| SBD-11 | EVQLVESGGGLVQPGGSLRLSCAVS | 79 | DIQMTQSPSSLSASVGDRVT | 80 |
| HC: G45C | GYSITSDYAWNWVRQAPGKCLEW | | ITCKSSQSLLYRSNQKNYLA | |
| LC: Q106C | VGYISNSGSTSYNPSLKSRFTISRDTS | | WYQQKPGKAPKLLIYWAS | |
| | KNTLYLQMNSLRAEDTAVYYCARE | | TRESGVPSRFSGSGSGTDFT | |
| | RNYDYDDYYYAMDYWGQGTLVT | | LTISSLQPEDFATYYCQQYY | |
| | VSS | | NYPRTFGCGTKVEIK | |
| SBD-12 | EVQLVESGGGLVQPGGSLRLSCAVS | 81 | DIQMTQSPSSLSASVGDRVT | 82 |
| HC: G45C | GYSITSDYAWNWVRQAPGKCLEW | | ITCKSSQSLLYRSNQKNYLA | |
| LC: Q106C | VGYISNSGSTSYNPSLKSRFTISRDTS | | WYQQKPGKAPKLLIYWAS | |
| | KNTLYLQMNSLRAEDTAVYYCARE | | TRESGVPSRFSGSGSGTDFT | |
| | RNYDYDDYYYAMDYWGQGTLVT | | LTISSLQPEDFATYYCQQYY | |
| | VSS | | NYPRTFGCGTKVEIK | |

TABLE 6
Anti-STEAP-1 Fab Heavy Chain and Light Chain Sequences

| Construct | | SEQ | | SEQ |
|---|---|---|---|---|
| Name/ | | ID | | ID |
| mutations | Heavy Chain | NO: | Light Chain | NO: |
| SBD-2/HC: | EVQLVESGGGLVQPGGSLRLSCAAS | 39 | DIVMTQSPDSLAVSLGERA | 40 |
| V24A, Y27F, | GFSITSDYAWNWVRQAPGKGLEWV | | TINCKSSQSLLYRSNQKNYL | |
| T58I, T74N | GYISNSGSISYNPSLKSRFTISRDNSK | | AWYQQKPGQPPKLLIYWAS | |
| LC: Q3V, | NTLYLQMNSLRAEDTAVYYCARER | | TRESGVPDRFSGSGSGTDFT | |
| S9D, S12A, | NYDYDDYYYAMDYWGQGTLVTVS | | LTISSLQAEDVAVYYCQQY | |
| A13V, V15L, | SASTKGPSVFPLAPSSKSTSGGTAAL | | YNYPRTFGQGTKVEIKRTV | |
| D17E, V19A, | GCLVKDYFPEPVTVSWNSGALTSG | | AAPSVFIFPPSDEQLKSGTA | |
| T22N, K48Q, | VHTFPAVLQSSGLYSLSSVVTVPSSS | | SVVCLLNNFYPREAKVQW | |
| A49P, S66D, | LGTQTYICNVNHKPSNTKVDKKVEP | | KVDNALQSGNSQESVTEQD | |
| P86A, F89V, | KSC | | SKDSTYSLSSTLTLSKADYE | |
| T91V | | | KHKVYACEVTHQGLSSPVT | |
| | | | KSFNRGEC | |
| SBD-3/HC: | EVQLVESGGGLVQPGGSLRLSCAVS | 41 | DIQMTQSPSSLSASVGDRVT | 42 |
| W35M, L64V | GYSITSDYAMNWVRQAPGKGLEW | | ITCKSSQSLLYRSNNKNYLA | |
| LC: Q35N | VGYISNSGSTSYNPSVKSRFTISRDT | | WYQQKPGKAPKLLIYWAS | |
| | SKNTLYLQMNSLRAEDTAVYYCAR | | TRESGVPSRFSGSGSGTDFT | |
| | ERNYDYDDYYYAMDYWGQGTLVT | | LTISSLQPEDFATYYCQQYY | |
| | VSSASTKGPSVFPLAPSSKSTSGGTA | | NYPRTFGQGTKVEIKRTVA | |
| | ALGCLVKDYFPEPVTVSWNSGALTS | | APSVFIFPPSDEQLKSGTAS | |
| | GVHTFPAVLQSSGLYSLSSVVTVPSS | | VVCLLNNFYPREAKVQWK | |
| | SLGTQTYICNVNHKPSNTKVDKKVE | | VDNALQSGNSQESVTEQDS | |
| | PKSC | | KDSTYSLSSTLTLSKADYEK | |
| | | | HKVYACEVTHQGLSSPVTK | |
| | | | SFNRGEC | |
| SBD-4/HC: | EVQLVESGGGLVQPGGSLRLSCAAS | 43 | DIVMTQSPDSLAVSLGERA | 44 |
| V24A, Y27F, | GFSITSDYAMNWVRQAPGKGLEWV | | TINCKSSQSLLYRSNNKNYL | |
| W35M, T58I, | GYISNSGSISYNPSVKGRFTISRDNS | | AWYQQKPGQPPKLLIYWAS | |
| L64V, S66G, | KNTLYLQMNSLRAEDTAVYYCARE | | TRESGVPDRFSGSGSGTDFT | |
| T74N | RNYDYDDYYYAMDYWGQGTLVT | | LTISSLQAEDVAVYYCQQY | |
| LC: Q3V, | VSSASTKGPSVFPLAPSSKSTSGGTA | | YNYPRTFGQGTKVEIKRTV | |
| S9D, S12A, | ALGCLVKDYFPEPVTVSWNSGALTS | | AAPSVFIFPPSDEQLKSGTA | |
| A13V, V15L, | GVHTFPAVLQSSGLYSLSSVVTVPSS | | SVVCLLNNFYPREAKVQW | |
| D17E, V19A, | SLGTQTYICNVNHKPSNTKVDKKVE | | KVDNALQSGNSQESVTEQD | |
| T22N, Q35N, | PKSC | | SKDSTYSLSSTLTLSKADYE | |
| K48Q, A49P, | | | KHKVYACEVTHQGLSSPVT | |
| S66D, P86A, | | | KSFNRGEC | |
| F89V, T91V | | | | |
| SBD-5/HC: | EVQLVESGGGLVQPGGSLRLSCAVS | 45 | DIQMTQSPSSLSASVGDRVT | 46 |
| W35M, L64V, | GYSITSDYAMNWVRQAPGKGLEW | | ITCKSSQSLLYRSNNKNYLA | |
| E99D, M111F | VGYISNSGSTSYNPSVKSRFTISRDT | | WYQQKPGKAPKLLIYWAS | |
| LC: Q35N | SKNTLYLQMNSLRAEDTAVYYCAR | | TRESGVPSRFSGSGSGTDFT | |
| | DRNYDYDDYYYAFDYWGQGTLVT | | LTISSLQPEDFATYYCQQYY | |
| | VSSASTKGPSVFPLAPSSKSTSGGTA | | NYPRTFGQGTKVEIKRTVA | |
| | ALGCLVKDYFPEPVTVSWNSGALTS | | APSVFIFPPSDEQLKSGTAS | |
| | GVHTFPAVLQSSGLYSLSSVVTVPSS | | VVCLLNNFYPREAKVQWK | |
| | SLGTQTYICNVNHKPSNTKVDKKVE | | VDNALQSGNSQESVTEQDS | |
| | PKSC | | KDSTYSLSSTLTLSKADYEK | |
| | | | HKVYACEVTHQGLSSPVTK | |
| | | | SFNRGEC | |
| SBD-6/HC: | EVQLVESGGGLVQPGGSLRLSCAAS | 47 | DIVMTQSPDSLAVSLGERA | 48 |
| V24A, Y27F, | GFSITSDYAMNWVRQAPGKGLEWV | | TINCKSSQSLLYRSNNKNYL | |
| W35M, L64V, | GYISNSGSTSYNPSVKSRFTISRDNS | | AWYQQKPGQPPKLLIYWAS | |
| T74N, E99D, | KNTLYLQMNSLRAEDTAVYYCARD | | TRESGVPDRFSGSGSGTDFT | |
| M111F | RNYDYDDYYYAFDYWGQGTLVTV | | LTISSLQAEDVAVYYCQQY | |
| LC: Q3V, | SSASTKGPSVFPLAPSSKSTSGGTAA | | YNYPRTFGQGTKVEIKRTV | |
| S9D, S12A, | LGCLVKDYFPEPVTVSWNSGALTSG | | AAPSVFIFPPSDEQLKSGTA | |
| A13V, V15L, | VHTFPAVLQSSGLYSLSSVVTVPSSS | | SVVCLLNNFYPREAKVQW | |
| D17E, V19A, | LGTQTYICNVNHKPSNTKVDKKVEP | | KVDNALQSGNSQESVTEQD | |
| T22N, Q35N, | KSC | | SKDSTYSLSSTLTLSKADYE | |
| K48Q, A49P, | | | KHKVYACEVTHQGLSSPVT | |
| S66D, P86A, | | | KSFNRGEC | |
| F89V, T91V | | | | |
| SBD-7/EE/RK | EVQLVESGGGLVQPGGSLRLSCAVS | 83 | DIQMTQSPSSLSASVGDRVT | 84 |
| HC: K154E, | GYSITSDYAWNWVRQAPGKGLEW | | ITCKSSQSLLYRSNQKNYLA | |
| K220E | VGYISNSGSTSYNPSLKSRFTISRDTS | | WYQQKPGKAPKLLIYWAS | |
| LC: E129R, | KNTLYLQMNSLRAEDTAVYYCARE | | TRESGVPSRFSGSGSGTDFT | |
| Q130K | RNYDYDDYYYAMDYWGQGTLVT | | LTISSLQPEDFATYYCQQYY | |
| | VSSASTKGPSVFPLAPSSKSTSGGTA | | NYPRTFGQGTKVEIKRTVA | |
| | ALGCLVEDYFPEPVTVSWNSGALTS | | APSVFIFPPSDRKLKSGTAS | |
| | GVHTFPAVLQSSGLYSLSSVVTVPSS | | VVCLLNNFYPREAKVQWK | |
| | SLGTQTYICNVNHKPSNGKVDEKVE | | VDNALQSGNSQESVTEQDS | |
| | PKSC | | KDSTYSLSSTLTLSKADYEK | |
| | | | HKVYACEVTHQGLSSPVTK | |
| | | | SFNRGEC | |
| SBD-8 | QVQLVQSGAEVKKPGASVKVSCKA | 85 | EIVLTQSPATLSLSPGERATL | 86 |
| | SGYTFSTYWIEWVRQAPGQRLEWM | | SCRASSSVSYMHWFQQKPG | |
| | GEILPGSGQTDFNEKFQGRVTFTAD | | QAPRLLIYSTSNLASGIPARF | |
| | TSSDTAYMELSSLRSEDTAVYYCTR | | SGSGSGTDYTLTISSLEPEDF | |
| | WGYYGTRGYFNVWGQGTLVTVSS | | AVYYCQQRRSFPYTFGQGT | |
| | ASTKGPSVFPLAPSSKSTSGGTAALG | | KLEIKRTVAAPSVFIFPPSDE | |
| | CLVKDYFPEPVTVSWNSGALTSGV | | QLKSGTASVVCLLNNFYPR | |
| | HTFPAVLQSSGLYSLSSVVTVPSSSL | | EAKVQWKVDNALQSGNSQ | |
| | GTQTYICNVNHKPSNTKVDKKVEP | | ESVTEQDSKDSTYSLSSTLT | |
| | KSC | | LSKADYEKHKVYACEVTH | |
| | | | QGLSSPVTKSFNRGEC | |

In some embodiments, the immunoglobulin heavy chain comprises the amino acid sequence of SEQ ID NO: 79 and the immunoglobulin light chain comprises the amino acid sequence of SEQ ID NO: 80.

In some embodiments, the antibody or antigen binding fragment thereof comprises the amino acid sequence of SEQ ID NO: 89. In some embodiments, the antibody or antigen binding fragment thereof comprises the amino acid sequence of SEQ ID NO: 90.

In some embodiments, the immunoglobulin heavy chain comprises amino acid mutations K154E and K220E, wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 90% sequence identity to SEQ ID NO: 83 and the immunoglobulin light chain comprises an amino acid sequence of at least 90% sequence identity to SEQ ID NO: 84. In some embodiments, the immunoglobulin heavy chain comprises amino acid mutations K154E and K220E, wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 92% sequence identity to SEQ ID NO: 83 and the immunoglobulin light chain comprises an amino acid sequence of at least 92% sequence identity to SEQ ID NO: 84. In some embodiments, the immunoglobulin heavy chain comprises amino acid mutations K154E and K220E, wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 95% sequence identity to SEQ ID NO: 83 and the immunoglobulin light chain comprises an amino acid sequence of at least 95% sequence identity to SEQ ID NO: 84.

In some embodiments, the immunoglobulin heavy chain comprises an amino acid sequence of at least 99% sequence identity to SEQ ID NO: 49 and the immunoglobulin light chain comprises an amino acid sequence of at least 99% sequence identity to SEQ ID NO: 50.

In some embodiments, the immunoglobulin heavy chain comprises an amino acid sequence of at least 95% sequence identity to SEQ ID NO: 51 and the immunoglobulin light chain comprises an amino acid sequence of at least 95% sequence identity to SEQ ID NO: 52. In some embodiments, the immunoglobulin heavy chain comprises an amino acid sequence of at least 99% sequence identity to SEQ ID NO: 51 and the immunoglobulin light chain comprises an amino acid sequence of at least 99% sequence identity to SEQ ID NO: 52. In some embodiments, the immunoglobulin heavy chain comprises the amino acid sequence of SEQ ID NO: 51 and the immunoglobulin light chain comprises the amino acid sequence of SEQ ID NO: 52.

In some embodiments, the immunoglobulin heavy chain comprises an amino acid sequence of at least 95% sequence identity to SEQ ID NO: 53 and the immunoglobulin light chain comprises an amino acid sequence of at least 95% sequence identity to SEQ ID NO: 54. In some embodiments, the immunoglobulin heavy chain comprises an amino acid sequence of at least 99% sequence identity to SEQ ID NO: 53 and the immunoglobulin light chain comprises an amino acid sequence of at least 99% sequence identity to SEQ ID NO: 54. In some embodiments, the immunoglobulin heavy chain comprises the amino acid sequence of SEQ ID NO: 53 and the immunoglobulin light chain comprises the amino acid sequence of SEQ ID NO: 54.

In some embodiments, the immunoglobulin heavy chain comprises an amino acid sequence of at least 95% sequence identity to SEQ ID NO: 55 and the immunoglobulin light chain comprises an amino acid sequence of at least 95% sequence identity to SEQ ID NO: 56. In some embodiments, the immunoglobulin heavy chain comprises an amino acid sequence of at least 99% sequence identity to SEQ ID NO: 55 and the immunoglobulin light chain comprises an amino acid sequence of at least 99% sequence identity to SEQ ID NO: 56. In some embodiments, the immunoglobulin heavy chain comprises the amino acid sequence of SEQ ID NO: 55 and the immunoglobulin light chain comprises the amino acid sequence of SEQ ID NO: 56.

In some embodiments, the immunoglobulin heavy chain comprises an amino acid sequence of at least 95% sequence identity to SEQ ID NO: 57 and the immunoglobulin light chain comprises an amino acid sequence of at least 95% sequence identity to SEQ ID NO: 58. In some embodiments, the immunoglobulin heavy chain comprises an amino acid sequence of at least 99% sequence identity to SEQ ID NO: 57 and the immunoglobulin light chain comprises an amino acid sequence of at least 99% sequence identity to SEQ ID NO: 58. In some embodiments, the immunoglobulin heavy chain comprises the amino acid sequence of SEQ ID NO: 57 and the immunoglobulin light chain comprises the amino acid sequence of SEQ ID NO: 58.

In some embodiments, the immunoglobulin heavy chain comprises an amino acid sequence of at least 95% sequence identity to SEQ ID NO: 59 and the immunoglobulin light chain comprises an amino acid sequence of at least 95% sequence identity to SEQ ID NO: 60. In some embodiments, the immunoglobulin heavy chain comprises an amino acid sequence of at least 99% sequence identity to SEQ ID NO: 59 and the immunoglobulin light chain comprises an amino acid sequence of at least 99% sequence identity to SEQ ID NO: 60. In some embodiments, the immunoglobulin heavy chain comprises the amino acid sequence of SEQ ID NO: 59 and the immunoglobulin light chain comprises the amino acid sequence of SEQ ID NO: 60.

TABLE 7
Anti-STEAP Heavy Chain and Light Chain IgG Sequences

| Construct | | SEQ | | SEQ |
|---|---|---|---|---|
| Name/ | | ID | | ID |
| mutations | Heavy Chain | NO: | Light Chain | NO: |
| IgG-1/ | EVQLVESGGGLVQPGGSLRLSCAVS | 49 | DIQMTQSPSSLSASVGDRVT | 50 |
| Wt | GYSITSDYAWNWVRQAPGKGLEW | | ITCKSSQSLLYRSNQKNYLA | |
| | VGYISNSGSTSYNPSLKSRFTISRDTS | | WYQQKPGKAPKLLIYWAS | |
| | KNTLYLQMNSLRAEDTAVYYCARE | | TRESGVPSRFSGSGSGTDFT | |
| | RNYDYDDYYYAMDYWGQGTLVT | | LTISSLQPEDFATYYCQQYY | |
| | VSSASTKGPSVFPLAPSSKSTSGGTA | | NYPRTFGQGTKVEIKRTVA | |
| | ALGCLVKDYFPEPVTVSWNSGALTS | | APSVFIFPPSDEQLKSGTAS | |
| | GVHTFPAVLQSSGLYSLSSVVTVPSS | | VVCLLNNFYPREAKVQWK | |
| | SLGTQTYICNVNHKPSNTKVDKKVE | | VDNALQSGNSQESVTEQDS | |
| | PKSCDKTHTCPPCPAPELLGGPSVFL | | KDSTYSLSSTLTLSKADYEK | |
| | FPPKPKDTLMISRTPEVTCVVVDVS | | HKVYACEVTHQGLSSPVTK | |
| | HEDPEVKFNWYVDGVEVHNAKTK | | SFNRGEC | |
| | PREEQYNSTYRVVSVLTVLHQDWL | | | |
| | NGKEYKCKVSNKALPAPIEKTISKA | | | |
| | KGQPREPQVYTLPPSRDELTKNQVS | | | |
| | LTCLVKGFYPSDIAVEWESNGQPEN | | | |
| | NYKTTPPVLDSDGSFFLYSKLTVDK | | | |
| | SRWQQGNVFSCSVMHEALHNHYT | | | |
| | QKSLSLSPGK | | | |
| IgG-2/HC: | EVQLVESGGGLVQPGGSLRLSCAAS | 51 | DIVMTQSPDSLAVSLGERA | 52 |
| V24A, Y27F, | GFSITSDYAWNWVRQAPGKGLEWV | | TINCKSSQSLLYRSNQKNYL | |
| T58I, T74N | GYISNSGSISYNPSLKSRFTISRDNSK | | AWYQQKPGQPPKLLIYWAS | |
| LC: Q3V, | NTLYLQMNSLRAEDTAVYYCARER | | TRESGVPDRFSGSGSGTDFT | |
| S9D, S12A, | NYDYDDYYYAMDYWGQGTLVTVS | | LTISSLQAEDVAVYYCQQY | |
| A13V, V15L, | SASTKGPSVFPLAPSSKSTSGGTAAL | | YNYPRTFGQGTKVEIKRTV | |
| D17E, V19A, | GCLVKDYFPEPVTVSWNSGALTSG | | AAPSVFIFPPSDEQLKSGTA | |
| T22N, K48Q, | VHTFPAVLQSSGLYSLSSVVTVPSSS | | SVVCLLNNFYPREAKVQW | |
| A49P, S66D, | LGTQTYICNVNHKPSNTKVDKKVEP | | KVDNALQSGNSQESVTEQD | |
| P86A, F89V, | KSCDKTHTCPPCPAPELLGGPSVFLF | | SKDSTYSLSSTLTLSKADYE | |
| T91V | PPKPKDTLMISRTPEVTCVVVDVSH | | KHKVYACEVTHQGLSSPVT | |
| | EDPEVKFNWYVDGVEVHNAKTKPR | | KSFNRGEC | |
| | EEQYNSTYRVVSVLTVLHQDWLNG | | | |
| | KEYKCKVSNKALPAPIEKTISKAKG | | | |
| | QPREPQVYTLPPSRDELTKNQVSLT | | | |
| | CLVKGFYPSDIAVEWESNGQPENNY | | | |
| | KTTPPVLDSDGSFFLYSKLTVDKSR | | | |
| | WQQGNVFSCSVMHEALHNHYTQK | | | |
| | SLSLSPGK | | | |
| IgG-3/HC: | EVQLVESGGGLVQPGGSLRLSCAVS | 53 | DIQMTQSPSSLSASVGDRVT | 54 |
| W35M, L64V | GYSITSDYAMNWVRQAPGKGLEW | | ITCKSSQSLLYRSNNKNYLA | |
| LC: Q35N | VGYISNSGSTSYNPSVKSRFTISRDT | | WYQQKPGKAPKLLIYWAS | |
| | SKNTLYLQMNSLRAEDTAVYYCAR | | TRESGVPSRFSGSGSGTDFT | |
| | ERNYDYDDYYYAMDYWGQGTLVT | | LTISSLQPEDFATYYCQQYY | |
| | VSSASTKGPSVFPLAPSSKSTSGGTA | | NYPRTFGQGTKVEIKRTVA | |
| | ALGCLVKDYFPEPVTVSWNSGALTS | | APSVFIFPPSDEQLKSGTAS | |
| | GVHTFPAVLQSSGLYSLSSVVTVPSS | | VVCLLNNFYPREAKVQWK | |
| | SLGTQTYICNVNHKPSNTKVDKKVE | | VDNALQSGNSQESVTEQDS | |
| | PKSCDKTHTCPPCPAPELLGGPSVFL | | KDSTYSLSSTLTLSKADYEK | |
| | FPPKPKDTLMISRTPEVTCVVVDVS | | HKVYACEVTHQGLSSPVTK | |
| | HEDPEVKFNWYVDGVEVHNAKTK | | SFNRGEC | |
| | PREEQYNSTYRVVSVLTVLHQDWL | | | |
| | NGKEYKCKVSNKALPAPIEKTISKA | | | |
| | KGQPREPQVYTLPPSRDELTKNQVS | | | |
| | LTCLVKGFYPSDIAVEWESNGQPEN | | | |
| | NYKTTPPVLDSDGSFFLYSKLTVDK | | | |
| | SRWQQGNVFSCSVMHEALHNHYT | | | |
| | QKSLSLSPGK | | | |
| IgG-4/HC: | EVQLVESGGGLVQPGGSLRLSCAAS | 55 | DIVMTQSPDSLAVSLGERA | 56 |
| V24A, Y27F, | GFSITSDYAMNWVRQAPGKGLEWV | | TINCKSSQSLLYRSNNKNYL | |
| W35M, T58I, | GYISNSGSISYNPSVKGRFTISRDNS | | AWYQQKPGQPPKLLIYWAS | |
| L64V, S66G, | KNTLYLQMNSLRAEDTAVYYCARE | | TRESGVPDRFSGSGSGTDFT | |
| T74N | RNYDYDDYYYAMDYWGQGTLVT | | LTISSLQAEDVAVYYCQQY | |
| LC: Q3V, | VSSASTKGPSVFPLAPSSKSTSGGTA | | YNYPRTFGQGTKVEIKRTV | |
| S9D, S12A, | ALGCLVKDYFPEPVTVSWNSGALTS | | AAPSVFIFPPSDEQLKSGTA | |
| A13V, V15L, | GVHTFPAVLQSSGLYSLSSVVTVPSS | | SVVCLLNNFYPREAKVQW | |
| D17E, V19A, | SLGTQTYICNVNHKPSNTKVDKKVE | | KVDNALQSGNSQESVTEQD | |
| T22N, Q35N, | PKSCDKTHTCPPCPAPELLGGPSVFL | | SKDSTYSLSSTLTLSKADYE | |
| K48Q, A49P, | FPPKPKDTLMISRTPEVTCVVVDVS | | KHKVYACEVTHQGLSSPVT | |
| S66D, P86A, | HEDPEVKFNWYVDGVEVHNAKTK | | KSFNRGEC | |
| F89V, T91V | PREEQYNSTYRVVSVLTVLHQDWL | | | |
| | NGKEYKCKVSNKALPAPIEKTISKA | | | |
| | KGQPREPQVYTLPPSRDELTKNQVS | | | |
| | LTCLVKGFYPSDIAVEWESNGQPEN | | | |
| | NYKTTPPVLDSDGSFFLYSKLTVDK | | | |
| | SRWQQGNVFSCSVMHEALHNHYT | | | |
| | QKSLSLSPGK | | | |
| IgG-5/HC: | EVQLVESGGGLVQPGGSLRLSCAVS | 57 | DIQMTQSPSSLSASVGDRVT | 58 |
| W35M, L64V, | GYSITSDYAMNWVRQAPGKGLEW | | ITCKSSQSLLYRSNNKNYLA | |
| E99D, M111F | VGYISNSGSTSYNPSVKSRFTISRDT | | WYQQKPGKAPKLLIYWAS | |
| LC: Q35N | SKNTLYLQMNSLRAEDTAVYYCAR | | TRESGVPSRFSGSGSGTDFT | |
| | DRNYDYDDYYYAFDYWGQGTLVT | | LTISSLQPEDFATYYCQQYY | |
| | VSSASTKGPSVFPLAPSSKSTSGGTA | | NYPRTFGQGTKVEIKRTVA | |
| | ALGCLVKDYFPEPVTVSWNSGALTS | | APSVFIFPPSDEQLKSGTAS | |
| | GVHTFPAVLQSSGLYSLSSVVTVPSS | | VVCLLNNFYPREAKVQWK | |
| | SLGTQTYICNVNHKPSNTKVDKKVE | | VDNALQSGNSQESVTEQDS | |
| | PKSCDKTHTCPPCPAPELLGGPSVFL | | KDSTYSLSSTLTLSKADYEK | |
| | FPPKPKDTLMISRTPEVTCVVVDVS | | HKVYACEVTHQGLSSPVTK | |
| | HEDPEVKFNWYVDGVEVHNAKTK | | SFNRGEC | |
| | PREEQYNSTYRVVSVLTVLHQDWL | | | |
| | NGKEYKCKVSNKALPAPIEKTISKA | | | |
| | KGQPREPQVYTLPPSRDELTKNQVS | | | |
| | LTCLVKGFYPSDIAVEWESNGQPEN | | | |
| | NYKTTPPVLDSDGSFFLYSKLTVDK | | | |
| | SRWQQGNVFSCSVMHEALHNHYT | | | |
| | QKSLSLSPGK | | | |
| IgG-6/HC: | EVQLVESGGGLVQPGGSLRLSCAAS | 59 | DIVMTQSPDSLAVSLGERA | 60 |
| V24A, Y27F, | GFSITSDYAMNWVRQAPGKGLEWV | | TINCKSSQSLLYRSNNKNYL | |
| W35M, L64V, | GYISNSGSTSYNPSVKSRFTISRDNS | | AWYQQKPGQPPKLLIYWAS | |
| T74N, E99D, | KNTLYLQMNSLRAEDTAVYYCARD | | TRESGVPDRFSGSGSGTDFT | |
| M111F | RNYDYDDYYYAFDYWGQGTLVTV | | LTISSLQAEDVAVYYCQQY | |
| LC: Q3V, | SSASTKGPSVFPLAPSSKSTSGGTAA | | YNYPRTFGQGTKVEIKRTV | |
| S9D, S12A, | LGCLVKDYFPEPVTVSWNSGALTSG | | AAPSVFIFPPSDEQLKSGTA | |
| A13V, V15L, | VHTFPAVLQSSGLYSLSSVVTVPSSS | | SVVCLLNNFYPREAKVQW | |
| D17E, V19A, | LGTQTYICNVNHKPSNTKVDKKVEP | | KVDNALQSGNSQESVTEQD | |
| T22N, Q35N, | KSCDKTHTCPPCPAPELLGGPSVFLF | | SKDSTYSLSSTLTLSKADYE | |
| K48Q, A49P, | PPKPKDTLMISRTPEVTCVVVDVSH | | KHKVYACEVTHQGLSSPVT | |
| S66D, P86A, | EDPEVKFNWYVDGVEVHNAKTKPR | | KSFNRGEC | |
| F89V, T91V | EEQYNSTYRVVSVLTVLHQDWLNG | | | |
| | KEYKCKVSNKALPAPIEKTISKAKG | | | |
| | QPREPQVYTLPPSRDELTKNQVSLT | | | |
| | CLVKGFYPSDIAVEWESNGQPENNY | | | |
| | KTTPPVLDSDGSFFLYSKLTVDKSR | | | |
| | WQQGNVFSCSVMHEALHNHYTQK | | | |
| | SLSLSPGK | | | |

TABLE 8
Anti-STEAP-1 scFv Sequences

| | | SEQ |
|---|---|---|
| Construct | | ID |
| Description | Amino Acid Sequence (N to C) | NO: |
| SBD-9 | EVQLVESGGGLVQPGGSLRLSCAVSGYSITSDYAWNWVRQAPGKGL | 87 |
| Vh-L-Vl | EWVGYISNSGSTSYNPSLKSRFTISRDTSKNTLYLQMNSLRAEDTA | |
| wt | VYYCARERNYDYDDYYYAMDYWGQGTLVTVSSGGGGSGGGGSGGGG | |
| | SDIQMTQSPSSLSASVGDRVTITCKSSQSLLYRSNQKNYLAWYQQK | |
| | PGKAPKLLIYWASTRESGVPSRFSGSGSGTDFTLTISSLQPEDFAT | |
| | YYCQQYYNYPRTFGQGTKVEIK | |
| SBD-10 | DIQMTQSPSSLSASVGDRVTITCKSSQSLLYRSNQKNYLAWYQQKP | 88 |
| Vl-L-Vh | GKAPKLLIYWASTRESGVPSRFSGSGSGTDFTLTISSLQPEDFATY | |
| wt | YCQQYYNYPRTFGQGTKVEIKGGGGSGGGGSGGGGSEVQLVESGGG | |
| | LVQPGGSLRLSCAVSGYSITSDYAWNWVRQAPGKGLEWVGYISNSG | |
| | STSYNPSLKSRFTISRDTSKNTLYLQMNSLRAEDTAVYYCARERNY | |
| | DYDDYYYAMDYWGQGTLVTVSS | |
| SBD-11 | EVQLVESGGGLVQPGGSLRLSCAVSGYSITSDYAWNWVRQAPGKCL | 89 |
| Vh-L-Vl | EWVGYISNSGSTSYNPSLKSRFTISRDTSKNTLYLQMNSLRAEDTA | |
| disulfide, | VYYCARERNYDYDDYYYAMDYWGQGTLVTVSSGGGGSGGGGSGGGG | |
| C44:C100 | SDIQMTQSPSSLSASVGDRVTITCKSSQSLLYRSNQKNYLAWYQQK | |
| | PGKAPKLLIYWASTRESGVPSRFSGSGSGTDFTLTISSLQPEDFAT | |
| | YYCQQYYNYPRTFGCGTKVEIK | |
| SBD-12 | DIQMTQSPSSLSASVGDRVTITCKSSQSLLYRSNQKNYLAWYQQKP | 90 |
| Vl-L-Vh, | GKAPKLLIYWASTRESGVPSRFSGSGSGTDFTLTISSLQPEDFATY | |
| Disulfide, | YCQQYYNYPRTFGCGTKVEIKGGGGSGGGGSGGGGSEVQLVESGGG | |
| C44:C100 | LVQPGGSLRLSCAVSGYSITSDYAWNWVRQAPGKCLEWVGYISNSG | |
| | STSYNPSLKSRFTISRDTSKNTLYLQMNSLRAEDTAVYYCARERNY | |
| | DYDDYYYAMDYWGQGTLVTVSS | |
| SBD-13 | QVQLVQSGAEVKKPGASVKVSCKASGYTFSTYWIEWVRQAPGQRLE | 105 |
| scFv | WMGEILPGSGQTDFNEKFQGRVTFTADTSSDTAYMELSSLRSEDTA | |
| (Vh-Vl) | VYYCTRWGYYGTRGYFNVWGQGTLVTVSSGGGGSGGGGSGGGGSEI | |
| | VLTQSPATLSLSPGERATLSCRASSSVSYMHWFQQKPGQAPRLLIY | |
| | STSNLASGIPARFSGSGSGTDYTLTISSLEPEDFAVYYCQQRRSFP | |
| | YTFGQGTKLEIK | |
| SBD-14 | EIVLTQSPATLSLSPGERATLSCRASSSVSYMHWFQQKPGQAPRLL | 106 |
| scFv | IYSTSNLASGIPARFSGSGSGTDYTLTISSLEPEDFAVYYCQQRRS | |
| (Vl-Vh) | FPYTFGQGTKLEIKGGGGSGGGGSGGGGSQVQLVQSGAEVKKPGAS | |
| | VKVSCKASGYTFSTYWIEWVRQAPGQRLEWMGEILPGSGQTDFNEK | |
| | FQGRVTFTADTSSDTAYMELSSLRSEDTAVYYCTRWGYYGTRGYFN | |
| | VWGQGTLVTVSS | |

In some embodiments, the polypeptide or polypeptide complex that comprises the antibody or antigen binding fragment thereof that binds to STEAP-1 is a monoclonal antibody. In some embodiments, the polypeptide or polypeptide complex that comprises the antibody or antigen binding fragment thereof that binds to STEAP-1 is a monospecific antibody, bispecific antibody, or trispecific antibody. In some embodiments, the antibody or antigen binding fragment thereof that binds to STEAP-1 comprises a Fab, Fab′, (Fab′)₂ or a single chain variable fragment (scFv). In some embodiments, the antibody or antigen binding fragment thereof that binds to STEAP-1 comprises the Fab. In some embodiments, the antibody or antigen binding fragment thereof that binds to STEAP-1 comprises the scFv. In some embodiments, a C-terminus of the immunoglobulin light chain of the scFv is connected to an N-terminus of the immunoglobulin heavy chain of the scFv via a linker (L). In some embodiments, a C-terminus of the immunoglobulin heavy chain of the scFv is connected to an N-terminus of the immunoglobulin light chain of the scFv via a linker (L).

In some embodiments, the polypeptide or polypeptide complex comprises more than one antibody or antigen binding fragment thereof that binds to STEAP-1. In some embodiments, the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L1). In some embodiments, L₁ connects an immunoglobulin heavy chain of the Fab or Fab′ that binds STEAP-1 with another immunoglobulin heavy chain of the second Fab or Fab′ that binds STEAP-1. In some embodiments, L₁ connects an immunoglobulin light chain of the Fab or Fab′ that binds STEAP-1 with another immunoglobulin light chain of the second Fab or Fab′ that binds STEAP-1. In some embodiments, L₁ connects an immunoglobulin light chain of the Fab or Fab′ that binds STEAP-1 with an immunoglobulin heavy chain of the second Fab or Fab′ that binds STEAP-1. In some embodiments, L or L₁ comprises the amino acid sequence selected from any one of SEQ ID NOs: 91-96 (see Table 9).

In some embodiments, the polypeptide or polypeptide complex comprises an antibody or antigen binding fragment thereof that binds to a second antigen other than STEAP-1.

TABLE 9
Linker Sequences

| Construct | Amino Acid Sequence | SEQ |
|---|---|---|
| Description | (N to C) | ID NO: |
| Linker | GGGGSGGGGSGGGGS | 91 |
| Linker | GGGGS | 92 |
| Linker | GGGGSGGGS | 93 |
| Linker | GGGGSGGGGS | 94 |
| Linker | GGGGSGGGGSGGGGSGGGGS | 95 |
| Linker | GGGGSGGGGSGGGGSGGGGSGGGGS | 96 |

### Modified Amino Acids

In some embodiments, the isolated polypeptide or polypeptide complex comprises a modified amino acid, a non-natural amino acid, a modified non-natural amino acid, or a combination thereof. In some embodiments, the non-natural amino acid comprises a D-amino acid. In some embodiments, the modified amino acid or modified non-natural amino acid comprises a post-translational modification.

### Pharmaceutical Compositions

Disclosed herein, in some embodiments, are pharmaceutical compositions comprising: (i) the polypeptide or polypeptide complex according to any embodiment disclosed herein, and (ii) a pharmaceutically acceptable excipient.

Disclosed herein, in some embodiments, are pharmaceutical compositions comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 61 and Q3, S9, S12, A13, V15, D17, V19, T22, Q35, K48, A49, S66, P86, F89, T91, and Q106 of SEQ ID NO: 62.

Disclosed herein, in some embodiments, are pharmaceutical compositions comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

In some embodiments, the polypeptide or polypeptide complex further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the polypeptide or polypeptide complex as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservatives, emulsifying agents and dispersing agents. Prevention of the action of microorganisms may be ensured by the inclusion of various antibacterial and antifungal agents.

The pharmaceutical composition may be in any suitable form, depending upon the desired method of administration. It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Isolated Recombinant Nucleic Acid Molecules

Disclosed herein, in some embodiments, are isolated recombinant nucleic acid molecules encoding a polypeptide of the antibody or antigen binding fragment thereof of any embodiment disclosed herein.

Disclosed herein, in some embodiments, are isolated recombinant nucleic acid molecules encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 61 and Q3, S9, S12, A13, V15, D17, V19, T22, Q35, K48, A49, S66, P86, F89, T91, and Q106 of SEQ ID NO: 62.

Disclosed herein, in some embodiments, are isolated recombinant nucleic acid molecules encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

### Methods of Treatment

Disclosed herein, in some embodiments, are methods of treating a cancer in a subject in need thereof comprising administering to the subject the polypeptide or polypeptide complex of any embodiment disclosed herein.

Disclosed herein, in some embodiments, are methods of treating cancer in a subject in need thereof comprising administering to the subject a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 61 and Q3, S9, S12, A13, V15, D17, V19, T22, Q35, K48, A49, S66, P86, F89, T91, and Q106 of SEQ ID NO: 62.

Disclosed herein, in some embodiments, are methods of treating cancer in a subject in need thereof comprising administering to the subject a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

In some embodiments, the cancer is sarcoma or a cancer of the prostate, breast, pancreas, bladder, gastrointestinal tract, testis, ovary, or cervix. In some embodiments, the cancer is prostate cancer or Ewing sarcoma.

In some embodiments, the method further comprises administering to the subject an immune checkpoint inhibitor. In some embodiments, the immune checkpoint inhibitor comprises an antibody or antigen binding fragment thereof that binds to PD-1.

### Production of Antibodies

In some embodiments, the antibodies or antigen binding fragments thereof as described herein are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding fragment is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

Disclosed herein, in some embodiments, are expression vectors comprising a nucleic acid encoding a polypeptide of the antibody or antigen binding fragment of any embodiment disclosed herein.

Disclosed herein, in some embodiments, are expression vectors comprising a nucleic acid encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 61 and Q3, S9, S12, A13, V15, D17, V19, T22, Q35, K48, A49, S66, P86, F89, T91, and Q106 of SEQ ID NO: 62.

Disclosed herein, in some embodiments are expression vectors comprising a nucleic acid encoding a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

In some embodiments, vectors include any suitable vectors derived from either eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

Disclosed herein, in some embodiments, are host cells comprising a polypeptide of the antibody or antigen binding fragment thereof of any embodiment disclosed herein.

Disclosed herein, in some embodiments, are host cells comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 61 and Q3, S9, S12, A13, V15, D17, V19, T22, Q35, K48, A49, S66, P86, F89, T91, and Q106 of SEQ ID NO: 62.

Disclosed herein, in some embodiments, are host cells comprising a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, and CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, and CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, and CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, and CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, and CDR3-L: SEQ ID NO: 38.

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVaF, TOP10/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVScl.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-KI cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a recombinant antibody or antigen binding fragment thereof of any embodiment disclosed herein.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Embodiments

Embodiment 1 comprises a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 61 and Q3, S9, S12, A13, V15, D17, V19, T22, Q35, K48, A49, S66, P86, F89, T91, and Q106 of SEQ ID NO: 62.

Embodiment 2 comprises the polypeptide or polypeptide complex of embodiment 1, wherein the at least one amino acid mutation is selected from V24A, Y27F, W35M, G45C, T58I, L64V, S66G, T74N, E99D, and M111F wherein the amino acid position corresponds to that of SEQ ID NO: 61 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62.

Embodiment 3 comprises the polypeptide or polypeptide complex of embodiments 1 or 2, wherein the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62.

Embodiment 4 comprises the polypeptide or polypeptide complex of any one of the preceding embodiments, wherein the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62, and the immunoglobulin heavy chain comprises an amino acid sequence of at least 90% sequence identity to SEQ ID NO: 61, and the immunoglobulin light chain comprises an amino acid sequence of at least 90% sequence identity to SEQ ID NO: 62.

Embodiment 5 comprises the polypeptide or polypeptide complex of any one of the preceding embodiments, wherein the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62, and the immunoglobulin heavy chain comprises an amino acid sequence of at least 95% sequence identity to SEQ ID NO: 61, and the immunoglobulin light chain comprises an amino acid sequence of at least 95% sequence identity to SEQ ID NO: 62.

Embodiment 6 comprises the polypeptide or polypeptide complex of any one of embodiments 1 to 5, wherein the immunoglobulin heavy chain comprises mutations V24A, Y27F, T58I, and T74N, and at least 90% sequence identity to SEQ ID NO: 61.

Embodiment 7 comprises the polypeptide or polypeptide complex of any one of embodiments 1 to 5, wherein the immunoglobulin heavy chain comprises mutations W35M and L64V and at least 90% sequence identity to SEQ ID NO: 61.

Embodiment 8 comprises the polypeptide or polypeptide complex of any one of embodiments 1 to 5, wherein the immunoglobulin heavy chain comprises mutations V24A, Y27F, W35M, T58I, L64V, S66G, and T74N and at least 90% sequence identity to SEQ ID NO: 61.

Embodiment 9 comprises the polypeptide or polypeptide complex of any one of embodiments 1 to 5, wherein the immunoglobulin heavy chain comprises mutations W35M, L64V, E99D, and M111F and at least 90% sequence identity to SEQ ID NO: 61.

Embodiment 10 comprises the polypeptide or polypeptide complex of any one of embodiments 1 to 5, wherein the immunoglobulin heavy chain comprises mutations V24A, Y27F, W35M, L64V, T74N, E99D, and M111F and at least 90% sequence identity to SEQ ID NO: 61.

Embodiment 11 comprises the polypeptide or polypeptide complex of any one of embodiments 1 to 5, wherein the immunoglobulin heavy chain comprises the G45C mutation and at least 90% sequence identity to SEQ ID NO: 61.

Embodiment 12 comprises the polypeptide or polypeptide complex of any one of embodiments 1 to 11, wherein the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 62.

Embodiment 13 comprises the polypeptide or polypeptide complex of any one of embodiments 1 to 11, wherein the immunoglobulin light chain comprises the Q35N mutation and at least 90% sequence identity to SEQ ID NO: 62.

Embodiment 14 comprises the polypeptide or polypeptide complex of any one of embodiments 1 to 11, wherein the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62.

Embodiment 15 comprises the polypeptide or polypeptide complex of any one of embodiments 1 to 11, wherein the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62.

Embodiment 16 comprises the polypeptide or polypeptide complex of any one of embodiments 1 to 11, wherein the immunoglobulin light chain comprises the Q106C mutation and at least 90% sequence identity to SEQ ID NO: 62.

Embodiment 17 comprises the polypeptide or polypeptide complex of any one of embodiments 1 to 5, wherein the immunoglobulin heavy chain comprises mutations V24A, Y27F, T58I, and T74N, and at least 90% sequence identity to SEQ ID NO: 61 and the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 62.

Embodiment 18 comprises the polypeptide or polypeptide complex of any one of embodiments 1 to 5, wherein the immunoglobulin heavy chain comprises mutations W35M and L64V and at least 90% sequence identity to SEQ ID NO: 61 and the immunoglobulin light chain comprises the Q35N mutation and at least 90% sequence identity to SEQ ID NO: 62.

Embodiment 19 comprises the polypeptide or polypeptide complex of any one of embodiments 1 to 5, wherein the immunoglobulin heavy chain comprises mutations V24A, Y27F, W35M, T58I, L64V, S66G, and T74N and at least 90% sequence identity to SEQ ID NO: 61 and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62.

Embodiment 20 comprises the polypeptide or polypeptide complex of any one of embodiments 1 to 5, wherein the immunoglobulin heavy chain comprises mutations W35M, L64V, E99D, and M111F and at least 90% sequence identity to SEQ ID NO: 61 and the immunoglobulin light chain comprises the Q35N mutation and at least 90% sequence identity to SEQ ID NO: 62.

Embodiment 21 comprises the polypeptide or polypeptide complex of any one of embodiments 1 to 5, wherein the immunoglobulin heavy chain comprises mutations V24A, Y27F, W35M, L64V, T74N, E99D, and M111F and at least 90% sequence identity to SEQ ID NO: 61 and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62.

Embodiment 22 comprises the polypeptide or polypeptide complex of any one of embodiments 1 to 5, wherein the immunoglobulin heavy chain comprises the G45C mutation and at least 90% sequence identity to SEQ ID NO: 61 and the immunoglobulin light chain comprises the Q106C mutation and at least 90% sequence identity to SEQ ID NO: 62.

Embodiment 23 comprises the polypeptide or polypeptide complex of embodiment any one of embodiments 1 to 5, wherein the antibody or antigen binding fragment thereof comprises an engineered disulfide bond between the immunoglobulin heavy chain and the immunoglobulin light chain.

Embodiment 24 comprises the polypeptide or polypeptide complex of embodiment 22, wherein the antibody or antigen binding fragment thereof comprises an engineered disulfide bond between the cysteine at the position of the G45C mutation and the cysteine at the position of the Q106C mutation.

Embodiment 25 comprises the polypeptide or polypeptide complex of any one of the preceding embodiments, wherein the immunoglobulin light chain comprises complementarity determining regions (CDRs) CDR1-L, CDR2-L, and CDR3-L, and the immunoglobulin heavy chain comprises CDRs: CDR1-H, CDR2-H, and CDR3-H, wherein at least one of the CDRs comprises the amino acid sequence selected from SEQ ID NOs: 3-38.

Embodiment 26 comprises the polypeptide or polypeptide complex of any one of the preceding embodiments, wherein CDR1-H comprises the amino acid sequence selected from SEQ ID NOs: 3-8; wherein CDR2-H comprises the amino acid sequence selected from SEQ ID NOs: 9-14; and wherein CDR3-H comprises the amino acid sequence selected from SEQ ID NOs: 15-20.

Embodiment 27 comprises the polypeptide or polypeptide complex of any one of the preceding embodiments, wherein CDR1-L comprises the amino acid sequence selected from SEQ ID NOs: 21-26; wherein CDR2-L comprises the amino acid sequence selected from SEQ ID NOs: 27-32; and wherein CDR3-L comprises the amino acid sequence selected from SEQ ID NOs: 33-38.

Embodiment 28 comprises the polypeptide or polypeptide complex of embodiment any one of the preceding embodiments, wherein CDR1-H comprises the amino acid sequence selected from SEQ ID NOs: 3-8; wherein CDR2-H comprises the amino acid sequence selected from SEQ ID NOs: 9-14; wherein CDR3-H comprises the amino acid sequence selected from SEQ ID NOs: 15-20; wherein CDR1-L comprises the amino acid sequence selected from SEQ ID NOs: 21-26; wherein CDR2-L comprises the amino acid sequence selected from SEQ ID NOs: 27-32; and wherein CDR3-L comprises the amino acid sequence selected from SEQ ID NOs: 33-38.

Embodiment 29 comprises the polypeptide or polypeptide complex of any one of embodiments 1 to 5 and 25 to 28, wherein the immunoglobulin heavy chain comprises the G45C mutation and the immunoglobulin light chain comprises the Q106C mutation, and wherein the polypeptide or polypeptide complex comprises an amino acid sequence having at least 95%, 98%, 99%, or 100% sequence identity to SEQ ID NO: 89.

Embodiment 30 comprises the polypeptide or polypeptide complex of any one of embodiments 1 to 5 and 25 to 28, wherein the immunoglobulin heavy chain comprises the G45C mutation and the immunoglobulin light chain comprises the Q106C mutation, and wherein the polypeptide or polypeptide complex comprises an amino acid sequence having at least 95%, 98%, 99%, or 100% sequence identity to SEQ ID NO: 90.

Embodiment 31 comprises a polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of: CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38.

Embodiment 32 comprises the polypeptide or polypeptide complex of embodiment 1, wherein the immunoglobulin heavy chain and the immunoglobulin light chain comprise a set of six CDR sequences selected from the group consisting of: CDR1-H: SEQ ID NO: 3, CDR2-H: SEQ ID NO: 9, CDR3-H: SEQ ID NO: 15, CDR1-L: SEQ ID NO: 21, CDR2-L: SEQ ID NO: 27, CDR3-L: SEQ ID NO: 33; CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35; CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36; CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37; and CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38.

Embodiment 33 comprises the polypeptide or polypeptide complex of any one of the preceding embodiments, wherein the immunoglobulin light chain comprises a variable domain of an immunoglobulin kappa (IgK) or immunoglobulin lambda (IgL) light chain.

Embodiment 34 comprises the polypeptide or polypeptide complex of any one of the preceding embodiments, wherein the immunoglobulin heavy chain comprises a constant domain derived from an IgG1, IgG2, IgG3, or IgG4 heavy chain.

Embodiment 35 comprises the polypeptide or polypeptide complex of embodiments 1 or 31, wherein the immunoglobulin light chain and the immunoglobulin heavy chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; and wherein the immunoglobulin heavy chain comprises an amino acid sequence of at least 95% sequence identity to SEQ ID NO: 69 and the immunoglobulin light chain comprises an amino acid sequence of at least 95% sequence identity to SEQ ID NO: 70.

Embodiment 36 comprises the polypeptide or polypeptide complex of embodiment 35, wherein the immunoglobulin heavy chain comprises an amino acid sequence of at least 99% sequence identity to SEQ ID NO: 69 and the immunoglobulin light chain comprises an amino acid sequence of at least 99% sequence identity to SEQ ID NO: 70.

Embodiment 37 comprises the polypeptide or polypeptide complex of embodiment 35, wherein the immunoglobulin heavy chain comprises the amino acid sequence of SEQ ID NO: 69 and the immunoglobulin light chain comprises the amino acid sequence of SEQ ID NO: 70.

Embodiment 38 comprises the polypeptide or polypeptide complex of embodiments 1 or 31, wherein the immunoglobulin light chain and the immunoglobulin heavy chain comprise a set of six CDR sequences of CDR1-H: SEQ ID NO: 4, CDR2-H: SEQ ID NO: 10, CDR3-H: SEQ ID NO: 16, CDR1-L: SEQ ID NO: 22, CDR2-L: SEQ ID NO: 28, CDR3-L: SEQ ID NO: 34; and wherein the immunoglobulin heavy chain comprises an amino acid sequence of at least 95% sequence identity to SEQ ID NO: 39 and the immunoglobulin light chain comprises an amino acid sequence of at least 95% sequence identity to SEQ ID NO: 40.

Embodiment 39 comprises the polypeptide or polypeptide complex of embodiment 38, wherein the immunoglobulin heavy chain comprises an amino acid sequence of at least 99% sequence identity to SEQ ID NO: 39 and the immunoglobulin light chain comprises an amino acid sequence of at least 99% sequence identity to SEQ ID NO: 40.

Embodiment 40 comprises the polypeptide or polypeptide complex of embodiment 38, wherein the immunoglobulin heavy chain comprises the amino acid sequence of SEQ ID NO: 39 and the immunoglobulin light chain comprises the amino acid sequence of SEQ ID NO: 40.

Embodiment 41 comprises the polypeptide or polypeptide complex of embodiments 1 or 31, wherein the immunoglobulin light chain and the immunoglobulin heavy chain comprise a set of six CDR sequences of CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35; and wherein the immunoglobulin heavy chain comprises an amino acid sequence of at least 95% sequence identity to SEQ ID NO: 71 and the immunoglobulin light chain comprises an amino acid sequence of at least 95% sequence identity to SEQ ID NO: 72.

Embodiment 42 comprises the polypeptide or polypeptide complex of embodiment 41, wherein the immunoglobulin heavy chain comprises an amino acid sequence of at least 99% sequence identity to SEQ ID NO: 71 and the immunoglobulin light chain comprises an amino acid sequence of at least 99% sequence identity to SEQ ID NO: 72.

Embodiment 43 comprises the polypeptide or polypeptide complex of embodiment 41, wherein the immunoglobulin heavy chain comprises the amino acid sequence of SEQ ID NO: 71 and the immunoglobulin light chain comprises the amino acid sequence of SEQ ID NO: 72.

Embodiment 44 comprises the polypeptide or polypeptide complex of embodiments 1 or 31, wherein the immunoglobulin light chain and the immunoglobulin heavy chain comprise a set of six CDR sequences of CDR1-H: SEQ ID NO: 5, CDR2-H: SEQ ID NO: 11, CDR3-H: SEQ ID NO: 17, CDR1-L: SEQ ID NO: 23, CDR2-L: SEQ ID NO: 29, CDR3-L: SEQ ID NO: 35; and wherein the immunoglobulin heavy chain comprises an amino acid sequence of at least 95% sequence identity to SEQ ID NO: 41 and the immunoglobulin light chain comprises an amino acid sequence of at least 95% sequence identity to SEQ ID NO: 42.

Embodiment 45 comprises the polypeptide or polypeptide complex of embodiment 44, wherein the immunoglobulin heavy chain comprises an amino acid sequence of at least 99% sequence identity to SEQ ID NO: 41 and the immunoglobulin light chain comprises an amino acid sequence of at least 99% sequence identity to SEQ ID NO: 42.

Embodiment 46 comprises the polypeptide or polypeptide complex of embodiment 44, wherein the immunoglobulin heavy chain comprises the amino acid sequence of SEQ ID NO: 41 and the immunoglobulin light chain comprises the amino acid sequence of SEQ ID NO: 42.

Embodiment 47 comprises the polypeptide or polypeptide complex of embodiments 1 or 31, wherein the immunoglobulin light chain and the immunoglobulin heavy chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36; and wherein the immunoglobulin heavy chain comprises an amino acid sequence of at least 95% sequence identity to SEQ ID NO: 73 and the immunoglobulin light chain comprises an amino acid sequence of at least 95% sequence identity to SEQ ID NO: 74.

Embodiment 48 comprises the polypeptide or polypeptide complex of embodiment 47, wherein the immunoglobulin heavy chain comprises an amino acid sequence of at least 99% sequence identity to SEQ ID NO: 73 and the immunoglobulin light chain comprises an amino acid sequence of at least 99% sequence identity to SEQ ID NO: 74.

Embodiment 49 comprises the polypeptide or polypeptide complex of embodiment 47, wherein the immunoglobulin heavy chain comprises the amino acid sequence of SEQ ID NO: 73 and the immunoglobulin light chain comprises the amino acid sequence of SEQ ID NO: 74.

Embodiment 50 comprises the polypeptide or polypeptide complex of embodiments 1 or 31, wherein the immunoglobulin light chain and the immunoglobulin heavy chain comprise a set of six CDR sequences of CDR1-H: SEQ ID NO: 6, CDR2-H: SEQ ID NO: 12, CDR3-H: SEQ ID NO: 18, CDR1-L: SEQ ID NO: 24, CDR2-L: SEQ ID NO: 30, CDR3-L: SEQ ID NO: 36; and wherein the immunoglobulin heavy chain comprises an amino acid sequence of at least 95% sequence identity to SEQ ID NO: 43 and the immunoglobulin light chain comprises an amino acid sequence of at least 95% sequence identity to SEQ ID NO: 44.

Embodiment 51 comprises the polypeptide or polypeptide complex of embodiment 50, wherein the immunoglobulin heavy chain comprises an amino acid sequence of at least 99% sequence identity to SEQ ID NO: 43 and the immunoglobulin light chain comprises an amino acid sequence of at least 99% sequence identity to SEQ ID NO: 44.

Embodiment 52 comprises the polypeptide or polypeptide complex of embodiment 50, wherein the immunoglobulin heavy chain comprises the amino acid sequence of SEQ ID NO: 43 and the immunoglobulin light chain comprises the amino acid sequence of SEQ ID NO: 44.

Embodiment 53 comprises the polypeptide or polypeptide complex of embodiments 1 or 31, wherein the immunoglobulin light chain and the immunoglobulin heavy chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37; and wherein the immunoglobulin heavy chain comprises an amino acid sequence of at least 95% sequence identity to SEQ ID NO: 75 and the immunoglobulin light chain comprises an amino acid sequence of at least 95% sequence identity to SEQ ID NO: 76.

Embodiment 54 comprises the polypeptide or polypeptide complex of embodiment 52, wherein the immunoglobulin heavy chain comprises an amino acid sequence of at least 99% sequence identity to SEQ ID NO: 75 and the immunoglobulin light chain comprises an amino acid sequence of at least 99% sequence identity to SEQ ID NO: 76.

Embodiment 55 comprises the polypeptide or polypeptide complex of embodiment 52, wherein the immunoglobulin heavy chain comprises the amino acid sequence of SEQ ID NO: 75 and the immunoglobulin light chain comprises the amino acid sequence of SEQ ID NO: 76.

Embodiment 56 comprises the polypeptide or polypeptide complex of embodiments 1 or 31, wherein the immunoglobulin light chain and the immunoglobulin heavy chain comprise a set of six CDR sequences of CDR1-H: SEQ ID NO: 7, CDR2-H: SEQ ID NO: 13, CDR3-H: SEQ ID NO: 19, CDR1-L: SEQ ID NO: 25, CDR2-L: SEQ ID NO: 31, CDR3-L: SEQ ID NO: 37; and wherein the immunoglobulin heavy chain comprises an amino acid sequence of at least 95% sequence identity to SEQ ID NO: 45 and the immunoglobulin light chain comprises an amino acid sequence of at least 95% sequence identity to SEQ ID NO: 46.

Embodiment 57 comprises the polypeptide or polypeptide complex of embodiment 56, wherein the immunoglobulin heavy chain comprises an amino acid sequence of at least 99% sequence identity to SEQ ID NO: 45 and the immunoglobulin light chain comprises an amino acid sequence of at least 99% sequence identity to SEQ ID NO: 46.

Embodiment 58 comprises the polypeptide or polypeptide complex of embodiment 57, wherein the immunoglobulin heavy chain comprises the amino acid sequence of SEQ ID NO: 45 and the immunoglobulin light chain comprises the amino acid sequence of SEQ ID NO: 46.

Embodiment 59 comprises the polypeptide or polypeptide complex of embodiments 1 or 31, wherein the immunoglobulin light chain and the immunoglobulin heavy chain comprise a set of six CDR sequences of CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38; and wherein the immunoglobulin heavy chain comprises an amino acid sequence of at least 95% sequence identity to SEQ ID NO: 77 and the immunoglobulin light chain comprises an amino acid sequence of at least 95% sequence identity to SEQ ID NO: 78.

Embodiment 60 comprises the polypeptide or polypeptide complex of embodiment 59, wherein the immunoglobulin heavy chain comprises an amino acid sequence of at least 99% sequence identity to SEQ ID NO: 77 and the immunoglobulin light chain comprises an amino acid sequence of at least 99% sequence identity to SEQ ID NO: 78.

Embodiment 61 comprises the polypeptide or polypeptide complex of embodiment 59, wherein the immunoglobulin heavy chain comprises the amino acid sequence of SEQ ID NO: 77 and the immunoglobulin light chain comprises the amino acid sequence of SEQ ID NO: 78.

Embodiment 62 comprises the polypeptide or polypeptide complex of embodiments 1 or 31, wherein the immunoglobulin light chain and the immunoglobulin heavy chain comprise a set of six CDR sequences of CDR1-H: SEQ ID NO: 8, CDR2-H: SEQ ID NO: 14, CDR3-H: SEQ ID NO: 20, CDR1-L: SEQ ID NO: 26, CDR2-L: SEQ ID NO: 32, CDR3-L: SEQ ID NO: 38; and wherein the immunoglobulin heavy chain comprises an amino acid sequence of at least 95% sequence identity to SEQ ID NO: 47 and the immunoglobulin light chain comprises an amino acid sequence of at least 95% sequence identity to SEQ ID NO: 48.

Embodiment 63 comprises the polypeptide or polypeptide complex of embodiment 62, wherein the immunoglobulin heavy chain comprises an amino acid sequence of at least 99% sequence identity to SEQ ID NO: 47 and the immunoglobulin light chain comprises an amino acid sequence of at least 99% sequence identity to SEQ ID NO: 48.

Embodiment 64 comprises the polypeptide or polypeptide complex of embodiment 62, wherein the immunoglobulin heavy chain comprises the amino acid sequence of SEQ ID NO: 47 and the immunoglobulin light chain comprises the amino acid sequence of SEQ ID NO: 48.

Embodiment 65 comprises the polypeptide or polypeptide complex of embodiments 1 or 31, wherein the immunoglobulin heavy chain comprises the amino acid sequence of SEQ ID NO: 79 and the immunoglobulin light chain comprises the amino acid sequence of SEQ ID NO: 80.

Embodiment 66 comprises the polypeptide or polypeptide complex of embodiments 1 or 31, wherein the antibody or antigen binding fragment thereof comprises the amino acid sequence of SEQ ID NO: 89.

Embodiment 67 comprises the polypeptide or polypeptide complex of embodiments 1 or 31, wherein the antibody or antigen binding fragment thereof comprises the amino acid sequence of SEQ ID NO: 90.

Embodiment 68 comprises the polypeptide or polypeptide complex of embodiments 1 or 31, wherein the immunoglobulin heavy chain comprises amino acid mutations K154E and K220E wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 90% sequence identity to SEQ ID NO: 83 and the immunoglobulin light chain comprises an amino acid sequence of at least 90% sequence identity to SEQ ID NO: 84.

Embodiment 69 comprises the polypeptide or polypeptide complex of embodiments 1 or 31, wherein the immunoglobulin heavy chain comprises amino acid mutations K154E and K220E wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 95% sequence identity to SEQ ID NO: 83 and the immunoglobulin light chain comprises an amino acid sequence of at least 95% sequence identity to SEQ ID NO: 84.

Embodiment 70 comprises the polypeptide or polypeptide complex of embodiments 1 or 31, wherein the immunoglobulin heavy chain comprises an amino acid sequence of at least 99% sequence identity to SEQ ID NO: 49 and the immunoglobulin light chain comprises an amino acid sequence of at least 99% sequence identity to SEQ ID NO: 50.

Embodiment 71 comprises the polypeptide or polypeptide complex of embodiments 1 or 31, wherein the immunoglobulin heavy chain comprises an amino acid sequence of at least 99% sequence identity to SEQ ID NO: 51 and the immunoglobulin light chain comprises an amino acid sequence of at least 99% sequence identity to SEQ ID NO: 52.

Embodiment 72 comprises the polypeptide or polypeptide complex of embodiments 1 or 31, wherein the immunoglobulin heavy chain comprises the amino acid sequence of SEQ ID NO: 51 and the immunoglobulin light chain comprises the amino acid sequence of SEQ ID NO: 52.

Embodiment 73 comprises the polypeptide or polypeptide complex of embodiments 1 or 31, wherein the immunoglobulin heavy chain comprises an amino acid sequence of at least 99% sequence identity to SEQ ID NO: 53 and the immunoglobulin light chain comprises an amino acid sequence of at least 99% sequence identity to SEQ ID NO: 54.

Embodiment 74 comprises the polypeptide or polypeptide complex of embodiments 1 or 31, wherein the immunoglobulin heavy chain comprises the amino acid sequence of SEQ ID NO: 53 and the immunoglobulin light chain comprises the amino acid sequence of SEQ ID NO: 54.

Embodiment 75 comprises the polypeptide or polypeptide complex of embodiments 1 or 31, wherein the immunoglobulin heavy chain comprises an amino acid sequence of at least 99% sequence identity to SEQ ID NO: 55 and the immunoglobulin light chain comprises an amino acid sequence of at least 99% sequence identity to SEQ ID NO: 56.

Embodiment 76 comprises the polypeptide or polypeptide complex of embodiments 1 or 31, wherein the immunoglobulin heavy chain comprises the amino acid sequence of SEQ ID NO: 55 and the immunoglobulin light chain comprises the amino acid sequence of SEQ ID NO: 56.

Embodiment 77 comprises the polypeptide or polypeptide complex of embodiments 1 or 31, wherein the immunoglobulin heavy chain comprises an amino acid sequence of at least 99% sequence identity to SEQ ID NO: 57 and the immunoglobulin light chain comprises an amino acid sequence of at least 99% sequence identity to SEQ ID NO: 58.

Embodiment 78 comprises the polypeptide or polypeptide complex of embodiments 1 or 31, wherein the immunoglobulin heavy chain comprises the amino acid sequence of SEQ ID NO: 57 and the immunoglobulin light chain comprises the amino acid sequence of SEQ ID NO: 58.

Embodiment 79 comprises the polypeptide or polypeptide complex of embodiments 1 or 31, wherein the immunoglobulin heavy chain comprises an amino acid sequence of at least 99% sequence identity to SEQ ID NO: 59 and the immunoglobulin light chain comprises an amino acid sequence of at least 99% sequence identity to SEQ ID NO: 60.

Embodiment 80 comprises the polypeptide or polypeptide complex of embodiments 1 or 31, wherein the immunoglobulin heavy chain comprises the amino acid sequence of SEQ ID NO: 59 and the immunoglobulin light chain comprises the amino acid sequence of SEQ ID NO: 60.

Embodiment 81 comprises the polypeptide or polypeptide complex of any one of the preceding embodiments, wherein the polypeptide or polypeptide complex that comprises the antibody or antigen binding fragment thereof that binds to STEAP-1 is a monoclonal antibody.

Embodiment 82 comprises the polypeptide or polypeptide complex of any one of the preceding embodiments, wherein the polypeptide or polypeptide complex that comprises the antibody or antigen binding fragment thereof that binds to STEAP-1 is a monospecific antibody, bispecific antibody, or trispecific antibody.

Embodiment 83 comprises the polypeptide or polypeptide complex of any one of the preceding embodiments, wherein the antibody or antigen binding fragment thereof that binds to STEAP-1 comprises a Fab, Fab′, (Fab′)2 or a single chain variable fragment (scFv).

Embodiment 84 comprises the polypeptide or polypeptide complex of embodiment 83, wherein the antibody or antigen binding fragment thereof that binds to STEAP-1 comprises the Fab.

Embodiment 85 comprises the polypeptide or polypeptide complex of embodiment 83, wherein the antibody or antigen binding fragment thereof that binds to STEAP-1 comprises the scFv.

Embodiment 86 comprises the polypeptide or polypeptide complex of embodiment 85, wherein a C-terminus of the immunoglobulin light chain of the scFv is connected to an N-terminus of the immunoglobulin heavy chain of the scFv via a linker (L).

Embodiment 87 comprises the polypeptide or polypeptide complex of embodiment 85, wherein a C-terminus of the immunoglobulin heavy chain of the scFv is connected to an N-terminus of the immunoglobulin light chain of the scFv via a linker (L).

Embodiment 88 comprises the polypeptide or polypeptide complex of any one of the preceding embodiments, wherein the polypeptide or polypeptide complex comprises more than one antibody or antigen binding fragment thereof that binds to STEAP-1.

Embodiment 89 comprises the polypeptide or polypeptide complex of any one of the preceding claims, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L1).

Embodiment 90 comprises the polypeptide or polypeptide complex of embodiment 89, wherein L₁ connects an immunoglobulin heavy chain of the Fab or Fab′ that binds STEAP-1 with another immunoglobulin heavy chain of the second Fab or Fab′ that binds STEAP-1.

Embodiment 91 comprises the polypeptide or polypeptide complex of embodiment 89, wherein L₁ connects an immunoglobulin light chain of the Fab or Fab′ that binds STEAP-1 with another immunoglobulin light chain of the second Fab or Fab′ that binds STEAP-1.

Embodiment 92 comprises the polypeptide or polypeptide complex of embodiment 89, wherein L₁ connects an immunoglobulin light chain of the Fab or Fab′ that binds STEAP-1 with an immunoglobulin heavy chain of the second Fab or Fab′ that binds STEAP-1.

Embodiment 93 comprises the polypeptide or polypeptide complex of any one of embodiments 86 and 89-92, wherein L or L₁ comprises and amino acid sequence selected from any one of SEQ ID NOs: 91-96.

Embodiment 94 comprises the polypeptide or polypeptide complex of any one of the preceding embodiments, wherein the polypeptide or polypeptide complex comprises an antibody or antigen binding fragment thereof that binds to a second antigen other than STEAP-1.

Embodiment 95 comprises a pharmaceutical composition comprising: (i) the polypeptide or polypeptide complex of any one of embodiments 1-94; and (ii) a pharmaceutically acceptable excipient.

Embodiment 96 comprises an isolated recombinant nucleic acid molecule encoding a polypeptide of the antibody or antigen binding fragment thereof of any one of embodiments 1-94.

Embodiment 97 comprises an expression vector comprising a nucleic acid encoding a polypeptide of the antibody or antigen binding fragment thereof of any one of embodiments 1-94.

Embodiment 98 comprises a host cell comprising a polypeptide of the antibody or antigen binding fragment thereof of any one of embodiments 1-94.

Embodiment 99 comprises a method of treating a cancer in a subject in need thereof comprising administering to the subject the polypeptide or polypeptide complex of any one of embodiments 1-94.

Embodiment 100 comprises the method of embodiment 99, wherein the cancer is sarcoma or a cancer of the prostate, breast, pancreas, bladder, gastrointestinal tract, testis, ovary, or cervix.

Embodiment 101 comprises the method of embodiment 99, wherein the cancer is prostate cancer or Ewing sarcoma.

Embodiment 102 comprises the method of embodiment 99, further comprising administering to the subject an immune checkpoint inhibitor.

Embodiment 103 comprises the method of embodiment 102, wherein the immune checkpoint inhibitor comprises an antibody or antigen binding fragment thereof that binds to PD-1.

### Examples

### Example 1: STEAP-1 Mutant Binding to STEAP-1 Expressing Cell Lines by Flow Cytometry

STEAP-1 antibody mutants are tested for their ability to bind STEAP-1 antigen expressed on prostate cancer cell lines, LNCaP, 22RV1, or VCAP. STEAP-1 antibody binders are titrated in buffer and incubated with STEAP-1 expressing cell lines. After washing the cells, bound STEAP-1 antibody binders are detected using a fluorescently labeled anti-human secondary antibody. After a final wash, cells are fixed and mean fluorescent intensity (MFI) signal is measured via flow cytometry. Concentrations required to achieve half maximal fluorescent signal are calculated and reported as EC₅₀s.

### Example 2: STEAP-1 Mutant Activity Against Tumor Cell Lines

STEAP-1 antibody mutants are evaluated in a functional in vitro tumor cell killing assay using STEAP-1 positive cancer cell lines LNCaP, VCaP, 2Rv1, C4-2B, A673, IM-95, or OE33. Tumor cell line STEAP-1 expression is quantified via flow cytometry using a STEAP-1 antibody labeled with phycoerythrin (PE) in a 1:1 antibody:PE molar ratio and compared to PE Quantibrite beads according to the manufacturer's instructions. Tumor cell killing is measured using an xCelligence real time analyzer from Agilent that relies on sensor impedance measurements (cell index) that increases as tumor cells adhere, spread, and expand on the surface of the sensor. Likewise, as the tumor cells are killed, the impedance decreases. Tumor cells are added to the wells and allowed to adhere overnight on a 96 well plate. The following day, STEAP-1 antibody mutants titrated in human serum supplemented medium along with CD8+ T cells are added to the wells. Cell index measurements are taken periodically over a set time period. The cell index times number of hours (tumor cell growth kinetics) is then plotted versus concentration of STEAP-1 mutant antibody, where the concentration of antibody required to reduce the tumor growth by 50% (IC₅₀) is calculated using Graphpad Prism software.

### Example 3: Manufacturability of STEAP-1 Binders Containing Mutations and an Engineered Disulfide Bond

Additional experiments are performed to evaluate the manufacturability of STEAP-1 binding domains after the introduction of mutations or an engineered disulfide bond into the STEAP-1 binding domain. The manufacturing process utilizes cation exchange chromatography for polishing post affinity chromatography steps. STEAP-1 binders are produced by transient expression in Chinese hamster ovary (CHO) cells. Conditioned medium from CHO cells expressing antibodies of interest are first purified using a AKTA Pure FPLC by affinity chromatography (AC) using CH1-XL resin with a pH 4 acidic elution step. Material eluted from the AC step is neutralized in 1M Tris followed by dilution in salt-free buffer at pH 6.0. Diluted material is then passed onto a cation exchange column followed by elution using a salt gradient (0-500 mM NaCl over 20 column volumes). Protein elution is monitored using UV absorbance and area under the curve (AUC) of distinct peaks is calculated using Unicorn software.

### Example 4: Material Properties Post-Processing of STEAP-1 Binders Containing an Engineered Disulfide Bond

Several experiments are conducted to assess various properties of the polished materials post-cation exchange chromatography polishing. Material properties are assessed at the following conditions:
1. Non-stressed (TO),
2. After 12 days at 25° C. (T12),
3. After 5 days at 40° C. (T5),
4. After 12 days at 40° C. (T12),
5. After 19 days at 40° C. (T19), and
6. After 5× freeze-thaw cycles (FT) to −70° C.

To induce temperature stress, samples are stored upright in tightly sealed glass vials (Agilent Technologies) sealed with a PFTE/red silicone septa (Agilent Technologies) in a temperature-controlled chamber operated at 25±5° C. or 40° C.±5° C., respectively.

To induce free-thaw stress, samples are subjected to 5 freeze-thaw cycles as follows: freezing consisting of storing samples at least 2 h at −70±10° C. Frozen samples are then thawed at room temperature for at least 2 h.

Specifically, studies are conducted to assess the hydrophobicity, charge variants, melting temperature, aggregation temperature, melting onset temperature, diameter, and uniformity of the STEAP-1 binders.

### Hydrophobicity

First, a study is conducted using analytical hydrophobic interaction chromatography (HIC) to measure the hydrophobicity of the exemplary STEAP-1 binders. Hydrophobicity is an important property for reducing aggregation overtime during manufacturing.

Analytical HIC is performed on an Agilent 1290 II system (Agilent Technologies, Santa Clara, CA) using a MABPac HIC-10 column (ThermoFisher Scientific, Waltham, MA) 4.6 mm×100 mm, 5 μm particle size. The column is operated at 30° C. with a flow rate of 1.0 mL/min. Samples are diluted 1:1 by volume with mobile phase A prior to injection, and the column load is 10 μg. The mobile phase consists of (A) 50 mM sodium phosphate, 2M ammonium sulfate, pH=6.5 (B) 50 mM sodium phosphate, pH=6.5. The linear gradient is set as follows: 0-2 min, 0% B; 2.0-2.5 min, 0-40% B; 2.5-20.5 min, 40-100% B, 20.5-24.0 min, 100% B; 24.0-24.1 min, 100-0% B and 24.1-31 min, 0% B. Eluting proteins are detected by UV spectroscopy at a wavelength of 214 nm. Data are processed using the Agilent OpenLab CDS software version 3.5.0 (Agilent Technologies).

### Charge Variants

Another study utilizes imaged capillary isoelectric focusing (icIEF) to analyze the charge variants of the exemplary STEAP-1 binders. iCIEF analysis is performed on a Maurice instrument equipped with an iCIEF cartridge (ProteinSimple, #PS-MC02-C). All samples are buffer exchanged into water using 10 kDa Amicon Ultra MWCO devices (Millipore), and the protein concentration is adjusted to 0.5 mg/mL. Twenty microliters of sample is mixed with 80 μl of iCIEF separation mix before sample analysis. The iCIEF separation mix is prepared fresh by mixing the following in a separate tube. Amounts provided are sufficient for 1 sample: 1% methyl cellulose (ProteinSimple #PS-MDKO1-C), 35 μl; SimpleSol (ProteinSimple #046-575), 20 μl; pI Marker 7.05 (ProteinSimple #046-032), 0.5 μl; pI Marker 10.17 (ProteinSimple #046-35), 0.5 μl; Pharmalyte 5-8 (Cytiva, PN: 17-0453-01), 1 μl; Pharmalyte 8-10.5 (Cytiva, PN: 17-0455-01), 1 μl; 500 mM arginine (ProteinSimple, #PS-MDKO1-C) 0.4 μl and ultrapure water, 19.6 μl. Sample analysis on the Maurice proceeds with the following method: Sample load by vacuum aspiration 90s, focusing for protein separation 1 min at 1500 V followed by 8 min at 3000 V. Focused proteins are detected by native fluorescence detection. Data are processed and analyzed using Compass software (Version 3.0.0, ProteinSimple).

### Purity

Another study utilizes non-reduced capillary gel electrophoresis sodium dodecyl sulfate (CE-SDS) analysis to measure formation of impurities of exemplary STEAP-1 binders under temperature stressed conditions. CE-SDS is performed on a Maurice instrument (ProteinSimple, San Jose, CA) equipped with a CE-SDS plus cartridge (Bio-Techne #PS-MC02-SP). CE-SDS Plus reagent kit (Bio-Techne #PS-MRK01-S), CE-SDS top running buffer (Bio-Techne #046-384), Maurice CE-SDS 25× Internal Standard (Bio-Techne #046-144) and CE-SDS Molecular Weight Markers (Bio-Techne #046-432) follow the instructions provided with the cartridge. All samples are diluted with lx sample buffer to 0.5 mg/mL. Fifty microliter of diluted sample is mixed with 2 μl internal standard and 2.5 μl of freshly prepared 250 mM iodoacetamide. Samples are incubated for 10 min at 70° C. and 50 μl of the resulting supernatant was transferred to a 96-multiwell plate for analysis. Electrophoresis on the Maurice proceeds with the following method: Samples are loaded electrokinetically for 20 s at 4600 V, and separation proceeds for 35 min at 5750 V. Proteins are detected by UV spectroscopy at 220 nm. Electropherograms are processed with Compass software (Version 3.0.0, ProteinSimple, San Jose, CA).

### Thermostability

Another study is conducted to assess the thermostability (e.g., melting temperature, aggregation temperature, and melting onset temperature) of the exemplary STEAP-1 binders, using differential scanning fluorometry (DSF) performed on an UNcle instrument (Unchained Labs).

To conduct this study, nine microliters of sample in its corresponding formulation buffer are loaded in triplicate in single-use Uni devices (Unchained labs, 201-1009) with sample concentrations ranging from 1-3 mg/mL. The excitation wavelength is 266 nm, and full-spectrum intrinsic fluorescence spectra are collected from 250-720 nm. The temperature is ramped from 15 to 95° C. with a thermal ramp of 0.5° C./minute. Data are processed with UNcle software v. 6.01 with the barycentric mean (BCM) method considering the wavelength range from 300-410 nm. Melting temperature (Tm) is defined as the maximum value of the first derivative of the BCM trace, while the melting onset temperature (Tonset) is defined at a 10% threshold from the baseline for the first derivative of the BCM trace. The aggregation temperature (Tagg) is determined from the static light scattering (SLS) trace at a 10% from baseline threshold.

### Diameter

Another study is conducted to assess the average diameter of the exemplary STEAP-1 binders via the UNcle instrument using dynamic light scattering (DLS). Proteins are sized with DLS at 15° C. before initiating the thermal ramp for DSF. The acquisition time for DLS is 5 s and an average of 4 measurements is reported. DLS data are processed with UNcle software v. 6.01.

### Uniformity

Appearance of the exemplary STEAP-1 binders is observed. Appearance testing is performed following the procedures outlined in USP <790> using a visual inspection station (Angstrom Supply) providing a black and white background with controlled lighting at 2000-3750 lux. Color and clarity observations are also recorded during appearance testing.

The concentration of the exemplary STEAP-1 binders is measured. Protein concentration is determined by UV absorption spectroscopy at 280 nm on a NanoDrop One (ThermoFisher Scientific) using the pedestal method (1 mm path length). The instrument is blanked with formulation buffer prior to sample analysis. All protein determinations are performed in triplicate with 2 μl sample. Extinction coefficients are calculated with Expasy ProtParam tool (Gasteiger et al., Springer, 2005) using the primary sequence and assuming all disulfide bonds are formed.

The protein size of the exemplary STEAP-1 binders are further analyzed using size exclusion chromatography high performance liquid chromatography (SEC-HPLC). SEC-HPLC is performed on an Agilent 1290 II instrument (Agilent Technologies) fitted with an advanced Bio SEC (Agilent Technologies) pre-column and an analytical SEC column, respectively. The precolumn is a 4.6×50 mm, 300A, #PL1580-1301 and the SEC column is 4.6×300 mm, 300 Å, #PL1580-5301. SEC analysis is performed under isocratic conditions at a column temperature of 28° C. The mobile phase, composed of 30 mM sodium acetate and 500 mM sodium chloride (pH 5.5), is delivered at a flow rate of 0.35 mL/min. The column load is 8 μg and chromatographic separation is monitored at 214 nm via UV detection. The total run time for the method is 19.5 minutes. Data are processed using the Agilent OpenLab CDS software version 3.5.0 (Agilent Technologies).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain and comprises at least one amino acid mutation wherein the at least one amino acid mutation is selected from a position corresponding to V24, Y27, W35, G45, T58, L64, S66, T74, E99, and M111 of SEQ ID NO: 61 and Q3, S9, S12, A13, V15, D17, V19, T22, Q35, K48, A49, S66, P86, F89, T91, and Q106 of SEQ ID NO: 62.

2. The polypeptide or polypeptide complex of claim 1, wherein the at least one amino acid mutation is selected from V24A, Y27F, W35M, G45C, T58I, L64V, S66G, T74N, E99D, and M111F wherein the amino acid position corresponds to that of SEQ ID NO: 61 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62.

3. The polypeptide or polypeptide complex of claim 1, wherein the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62.

4. The polypeptide or polypeptide complex of claim 1, wherein the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62, and the immunoglobulin heavy chain comprises an amino acid sequence of at least 90% sequence identity to SEQ ID NO: 61, and the immunoglobulin light chain comprises an amino acid sequence of at least 90% sequence identity to SEQ ID NO: 62.

5. The polypeptide or polypeptide complex of claim 1, wherein the at least one amino acid mutation comprises at least two, at least three, at least four, at least five, at least ten, at least 15, or at least 20 mutations selected from V24A, Y27F, W35M, G45C, T58I, L64V, S66G, T74N, E99D, and M111F wherein the amino acid position corresponds to that of SEQ ID NO: 61 and Q3V, S9D, S12A, A13V, V15L, D17E, V19A, T22N, Q35N, K48Q, A49P, S66D, P86A, F89V, T91V, and Q106C wherein the amino acid position corresponds to that of SEQ ID NO: 62, and the immunoglobulin heavy chain comprises an amino acid sequence of at least 95% sequence identity to SEQ ID NO: 61, and the immunoglobulin light chain comprises an amino acid sequence of at least 95% sequence identity to SEQ ID NO: 62.

6. The polypeptide or polypeptide complex of claim 1, wherein the immunoglobulin heavy chain comprises mutations V24A, Y27F, T58I, and T74N, and at least 90% sequence identity to SEQ ID NO: 61.

7. The polypeptide or polypeptide complex of claim 1, wherein the immunoglobulin heavy chain comprises mutations W35M and L64V and at least 90% sequence identity to SEQ ID NO: 61.

8. The polypeptide or polypeptide complex of claim 1, wherein the immunoglobulin heavy chain comprises mutations V24A, Y27F, W35M, T58I, L64V, S66G, and T74N and at least 90% sequence identity to SEQ ID NO: 61.

9. The polypeptide or polypeptide complex of claim 1, wherein the immunoglobulin heavy chain comprises mutations W35M, L64V, E99D, and M111F and at least 90% sequence identity to SEQ ID NO: 61.

10. The polypeptide or polypeptide complex of claim 1, wherein the immunoglobulin heavy chain comprises mutations V24A, Y27F, W35M, L64V, T74N, E99D, and M111F and at least 90% sequence identity to SEQ ID NO: 61.

11. The polypeptide or polypeptide complex of claim 1, wherein the immunoglobulin heavy chain comprises a G45C mutation and at least 90% sequence identity to SEQ ID NO: 61.

12. The polypeptide or polypeptide complex of claim 1, wherein the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 62.

13. The polypeptide or polypeptide complex of claim 1, wherein the immunoglobulin light chain comprises a Q35N mutation and at least 90% sequence identity to SEQ ID NO: 62.

14. The polypeptide or polypeptide complex of claim 1, wherein the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62.

15. The polypeptide or polypeptide complex of any one of claims 1 to 11, wherein the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62.

16. The polypeptide or polypeptide complex of claim 1, wherein the immunoglobulin light chain comprises a Q106C mutation and at least 90% sequence identity to SEQ ID NO: 62.

17. The polypeptide or polypeptide complex of claim 1, wherein the immunoglobulin heavy chain comprises mutations V24A, Y27F, T58I, and T74N, and at least 90% sequence identity to SEQ ID NO: 61 and the immunoglobulin light chain comprises mutations Q3V, S9D, S12A, A13V, V15L, D17E, V19A, T22N, K48Q, A49P, S66D, P86A, F89V, and T91V and at least 90% sequence identity to SEQ ID NO: 62.

18. The polypeptide or polypeptide complex of claim 1, wherein the immunoglobulin heavy chain comprises mutations W35M and L64V and at least 90% sequence identity to SEQ ID NO: 61 and the immunoglobulin light chain comprises a Q35N mutation and at least 90% sequence identity to SEQ ID NO: 62.

19. The polypeptide or polypeptide complex of claim 1, wherein the immunoglobulin heavy chain comprises mutations V24A, Y27F, W35M, T58I, L64V, S66G, and T74N and at least 90% sequence identity to SEQ ID NO: 61 and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62.

20. The polypeptide or polypeptide complex of claim 1, wherein the immunoglobulin heavy chain comprises mutations W35M, L64V, E99D, and M111F and at least 90% sequence identity to SEQ ID NO: 61 and the immunoglobulin light chain comprises a Q35N mutation and at least 90% sequence identity to SEQ ID NO: 62.

21. The polypeptide or polypeptide complex of claim 1, wherein the immunoglobulin heavy chain comprises mutations V24A, Y27F, W35M, L64V, T74N, E99D, and M111F and at least 90% sequence identity to SEQ ID NO: 61 and the immunoglobulin light chain comprises mutations Q3V, S9D, S12A, A13V, V15L, D17E, V19A, T22N, Q35N, K48Q, A49P, S66D, P86A, F89V, and T91V and at least 90% sequence identity to SEQ ID NO: 62.

22. The polypeptide or polypeptide complex of claim 1, wherein the immunoglobulin heavy chain comprises a G45C mutation and at least 90% sequence identity to SEQ ID NO: 61 and the immunoglobulin light chain comprises a Q106C mutation and at least 90% sequence identity to SEQ ID NO: 62.

23. The polypeptide or polypeptide complex of claim 1, wherein the antibody or antigen binding fragment thereof comprises an engineered disulfide bond between the immunoglobulin heavy chain and the immunoglobulin light chain.

24. The polypeptide or polypeptide complex of claim 22, wherein the antibody or antigen binding fragment thereof comprises an engineered disulfide bond between the cysteine at the position of the G45C mutation and the cysteine at the position of the Q106C mutation.

25. The polypeptide or polypeptide complex of claim 1, wherein the immunoglobulin light chain comprises complementarity determining regions (CDRs) CDR1-L, CDR2-L, and CDR3-L, and the immunoglobulin heavy chain comprises CDRs: CDR1-H, CDR2-H, and CDR3-H, wherein at least one of the CDRs comprises the amino acid sequence selected from SEQ ID NOs: 3-38.

26. The polypeptide or polypeptide complex of claim 25,
wherein CDR1-H comprises the amino acid sequence selected from SEQ ID NOs: 3-8;
wherein CDR2-H comprises the amino acid sequence selected from SEQ ID NOs: 9-14; and
wherein CDR3-H comprises the amino acid sequence selected from SEQ ID NOs: 15-20.

27. The polypeptide or polypeptide complex of claim 25,
wherein CDR1-L comprises the amino acid sequence selected from SEQ ID NOs: 21-26;
wherein CDR2-L comprises the amino acid sequence selected from SEQ ID NOs: 27-32; and
wherein CDR3-L comprises the amino acid sequence selected from SEQ ID NOs: 33-38.

28. The polypeptide or polypeptide complex of claim 25,
wherein CDR1-H comprises the amino acid sequence selected from SEQ ID NOs: 3-8;
wherein CDR2-H comprises the amino acid sequence selected from SEQ ID NOs: 9-14;
wherein CDR3-H comprises the amino acid sequence selected from SEQ ID NOs: 15-20;
wherein CDR1-L comprises the amino acid sequence selected from SEQ ID NOs: 21-26;
wherein CDR2-L comprises the amino acid sequence selected from SEQ ID NOs: 27-32; and
wherein CDR3-L comprises the amino acid sequence selected from SEQ ID NOs: 33-38.

29. The polypeptide or polypeptide complex of claim 1, wherein the immunoglobulin heavy chain comprises a G45C mutation and the immunoglobulin light chain comprises a Q106C mutation, and wherein the polypeptide or polypeptide complex comprises an amino acid sequence having at least 95%, 98%, 99%, or 100% sequence identity to SEQ ID NO: 89.

30. The polypeptide or polypeptide complex of claim 1, wherein the immunoglobulin heavy chain comprises a G45C mutation and the immunoglobulin light chain comprises a Q106C mutation, and wherein the polypeptide or polypeptide complex comprises an amino acid sequence having at least 95%, 98%, 99%, or 100% sequence identity to SEQ ID NO: 90.

31. A polypeptide or polypeptide complex comprising an antibody or antigen binding fragment thereof that binds to six-transmembrane epithelial antigen of the prostate-1 (STEAP-1), wherein the antibody or antigen binding fragment thereof comprises an immunoglobulin light chain and an immunoglobulin heavy chain wherein the immunoglobulin heavy chain and the immunoglobulin light chain comprise a set of six CDR sequences selected from the group consisting of:
CDR1-H: SEQ ID NO: 4, CDR2-H: SEQ ID NO: 10, CDR3-H: SEQ ID NO: 16,
CDR1-L: SEQ ID NO: 22, CDR2-L: SEQ ID NO: 28, CDR3-L: SEQ ID NO: 34;
CDR1-H: SEQ ID NO: 5, CDR2-H: SEQ ID NO: 11, CDR3-H: SEQ ID NO: 17,
CDR1-L: SEQ ID NO: 23, CDR2-L: SEQ ID NO: 29, CDR3-L: SEQ ID NO: 35;
CDR1-H: SEQ ID NO: 6, CDR2-H: SEQ ID NO: 12, CDR3-H: SEQ ID NO: 18,
CDR1-L: SEQ ID NO: 24, CDR2-L: SEQ ID NO: 30, CDR3-L: SEQ ID NO: 36;
CDR1-H: SEQ ID NO: 7, CDR2-H: SEQ ID NO: 13, CDR3-H: SEQ ID NO: 19,
CDR1-L: SEQ ID NO: 25, CDR2-L: SEQ ID NO: 31, CDR3-L: SEQ ID NO: 37; and
CDR1-H: SEQ ID NO: 8, CDR2-H: SEQ ID NO: 14, CDR3-H: SEQ ID NO: 20,
CDR1-L: SEQ ID NO: 26, CDR2-L: SEQ ID NO: 32, CDR3-L: SEQ ID NO: 38.

32. The polypeptide or polypeptide complex of claim 1, wherein the immunoglobulin heavy chain and the immunoglobulin light chain comprise a set of six CDR sequences selected from the group consisting of:
CDR1-H: SEQ ID NO: 3, CDR2-H: SEQ ID NO: 9, CDR3-H: SEQ ID NO: 15,
CDR1-L: SEQ ID NO: 21, CDR2-L: SEQ ID NO: 27, CDR3-L: SEQ ID NO: 33;
CDR1-H: SEQ ID NO: 4, CDR2-H: SEQ ID NO: 10, CDR3-H: SEQ ID NO: 16,
CDR1-L: SEQ ID NO: 22, CDR2-L: SEQ ID NO: 28, CDR3-L: SEQ ID NO: 34;
CDR1-H: SEQ ID NO: 5, CDR2-H: SEQ ID NO: 11, CDR3-H: SEQ ID NO: 17,
CDR1-L: SEQ ID NO: 23, CDR2-L: SEQ ID NO: 29, CDR3-L: SEQ ID NO: 35;
CDR1-H: SEQ ID NO: 6, CDR2-H: SEQ ID NO: 12, CDR3-H: SEQ ID NO: 18,
CDR1-L: SEQ ID NO: 24, CDR2-L: SEQ ID NO: 30, CDR3-L: SEQ ID NO: 36;
CDR1-H: SEQ ID NO: 7, CDR2-H: SEQ ID NO: 13, CDR3-H: SEQ ID NO: 19,
CDR1-L: SEQ ID NO: 25, CDR2-L: SEQ ID NO: 31, CDR3-L: SEQ ID NO: 37; and
CDR1-H: SEQ ID NO: 8, CDR2-H: SEQ ID NO: 14, CDR3-H: SEQ ID NO: 20,
CDR1-L: SEQ ID NO: 26, CDR2-L: SEQ ID NO: 32, CDR3-L: SEQ ID NO: 38.

33. The polypeptide or polypeptide complex of claim 1, wherein the immunoglobulin light chain comprises a variable domain of an immunoglobulin kappa (IgK) or immunoglobulin lambda (IgL) light chain.

34. The polypeptide or polypeptide complex of claim 1, wherein the immunoglobulin heavy chain comprises a constant domain derived from an IgG1, IgG2, IgG3, or IgG4 heavy chain.

35. The polypeptide or polypeptide complex of claim 31, wherein the immunoglobulin light chain and the immunoglobulin heavy chain comprise a set of six CDR sequences of
CDR1-H: SEQ ID NO: 4, CDR2-H: SEQ ID NO: 10, CDR3-H: SEQ ID NO: 16,
CDR1-L: SEQ ID NO: 22, CDR2-L: SEQ ID NO: 28, CDR3-L: SEQ ID NO: 34;
and wherein the immunoglobulin heavy chain comprises an amino acid sequence of at least 95% sequence identity to SEQ ID NO: 69 and the immunoglobulin light chain comprises an amino acid sequence of at least 95% sequence identity to SEQ ID NO: 70.

36. The polypeptide or polypeptide complex of claim 35, wherein the immunoglobulin heavy chain comprises an amino acid sequence of at least 99% sequence identity to SEQ ID NO: 69 and the immunoglobulin light chain comprises an amino acid sequence of at least 99% sequence identity to SEQ ID NO: 70.

37. The polypeptide or polypeptide complex of claim 35, wherein the immunoglobulin heavy chain comprises the amino acid sequence of SEQ ID NO: 69 and the immunoglobulin light chain comprises the amino acid sequence of SEQ ID NO: 70.

38. The polypeptide or polypeptide complex of claim 31, wherein the immunoglobulin light chain and the immunoglobulin heavy chain comprise a set of six CDR sequences of
CDR1-H: SEQ ID NO: 4, CDR2-H: SEQ ID NO: 10, CDR3-H: SEQ ID NO: 16,
CDR1-L: SEQ ID NO: 22, CDR2-L: SEQ ID NO: 28, CDR3-L: SEQ ID NO: 34;
and wherein the immunoglobulin heavy chain comprises an amino acid sequence of at least 95% sequence identity to SEQ ID NO: 39 and the immunoglobulin light chain comprises an amino acid sequence of at least 95% sequence identity to SEQ ID NO: 40.

39. The polypeptide or polypeptide complex of claim 38, wherein the immunoglobulin heavy chain comprises an amino acid sequence of at least 99% sequence identity to SEQ ID NO: 39 and the immunoglobulin light chain comprises an amino acid sequence of at least 99% sequence identity to SEQ ID NO: 40.

40. The polypeptide or polypeptide complex of claim 38, wherein the immunoglobulin heavy chain comprises the amino acid sequence of SEQ ID NO: 39 and the immunoglobulin light chain comprises the amino acid sequence of SEQ ID NO: 40.

41. The polypeptide or polypeptide complex of claim 31, wherein the immunoglobulin light chain and the immunoglobulin heavy chain comprise a set of six CDR sequences of
CDR1-H: SEQ ID NO: 5, CDR2-H: SEQ ID NO: 11, CDR3-H: SEQ ID NO: 17,
CDR1-L: SEQ ID NO: 23, CDR2-L: SEQ ID NO: 29, CDR3-L: SEQ ID NO: 35;
and wherein the immunoglobulin heavy chain comprises an amino acid sequence of at least 95% sequence identity to SEQ ID NO: 71 and the immunoglobulin light chain comprises an amino acid sequence of at least 95% sequence identity to SEQ ID NO: 72.

42. The polypeptide or polypeptide complex of claim 41, wherein the immunoglobulin heavy chain comprises an amino acid sequence of at least 99% sequence identity to SEQ ID NO: 71 and the immunoglobulin light chain comprises an amino acid sequence of at least 99% sequence identity to SEQ ID NO: 72.

43. The polypeptide or polypeptide complex of claim 41, wherein the immunoglobulin heavy chain comprises the amino acid sequence of SEQ ID NO: 71 and the immunoglobulin light chain comprises the amino acid sequence of SEQ ID NO: 72.

44. The polypeptide or polypeptide complex of claim 31, wherein the immunoglobulin light chain and the immunoglobulin heavy chain comprise a set of six CDR sequences of
CDR1-H: SEQ ID NO: 5, CDR2-H: SEQ ID NO: 11, CDR3-H: SEQ ID NO: 17,
CDR1-L: SEQ ID NO: 23, CDR2-L: SEQ ID NO: 29, CDR3-L: SEQ ID NO: 35;
and wherein the immunoglobulin heavy chain comprises an amino acid sequence of at least 95% sequence identity to SEQ ID NO: 41 and the immunoglobulin light chain comprises an amino acid sequence of at least 95% sequence identity to SEQ ID NO: 42.

45. The polypeptide or polypeptide complex of claim 44, wherein the immunoglobulin heavy chain comprises an amino acid sequence of at least 99% sequence identity to SEQ ID NO: 41 and the immunoglobulin light chain comprises an amino acid sequence of at least 99% sequence identity to SEQ ID NO: 42.

46. The polypeptide or polypeptide complex of claim 44, wherein the immunoglobulin heavy chain comprises the amino acid sequence of SEQ ID NO: 41 and the immunoglobulin light chain comprises the amino acid sequence of SEQ ID NO: 42.

47. The polypeptide or polypeptide complex of claim 31, wherein the immunoglobulin light chain and the immunoglobulin heavy chain comprise a set of six CDR sequences of
CDR1-H: SEQ ID NO: 6, CDR2-H: SEQ ID NO: 12, CDR3-H: SEQ ID NO: 18,
CDR1-L: SEQ ID NO: 24, CDR2-L: SEQ ID NO: 30, CDR3-L: SEQ ID NO: 36;
and wherein the immunoglobulin heavy chain comprises an amino acid sequence of at least 95% sequence identity to SEQ ID NO: 73 and the immunoglobulin light chain comprises an amino acid sequence of at least 95% sequence identity to SEQ ID NO: 74.

48. The polypeptide or polypeptide complex of claim 47, wherein the immunoglobulin heavy chain comprises an amino acid sequence of at least 99% sequence identity to SEQ ID NO: 73 and the immunoglobulin light chain comprises an amino acid sequence of at least 99% sequence identity to SEQ ID NO: 74.

49. The polypeptide or polypeptide complex of claim 47, wherein the immunoglobulin heavy chain comprises the amino acid sequence of SEQ ID NO: 73 and the immunoglobulin light chain comprises the amino acid sequence of SEQ ID NO: 74.

50. The polypeptide or polypeptide complex of claim 31, wherein the immunoglobulin light chain and the immunoglobulin heavy chain comprise a set of six CDR sequences of
CDR1-H: SEQ ID NO: 6, CDR2-H: SEQ ID NO: 12, CDR3-H: SEQ ID NO: 18,
CDR1-L: SEQ ID NO: 24, CDR2-L: SEQ ID NO: 30, CDR3-L: SEQ ID NO: 36;
and wherein the immunoglobulin heavy chain comprises an amino acid sequence of at least 95% sequence identity to SEQ ID NO: 43 and the immunoglobulin light chain comprises an amino acid sequence of at least 95% sequence identity to SEQ ID NO: 44.

51. The polypeptide or polypeptide complex of claim 50, wherein the immunoglobulin heavy chain comprises an amino acid sequence of at least 99% sequence identity to SEQ ID NO: 43 and the immunoglobulin light chain comprises an amino acid sequence of at least 99% sequence identity to SEQ ID NO: 44.

52. The polypeptide or polypeptide complex of claim 50, wherein the immunoglobulin heavy chain comprises the amino acid sequence of SEQ ID NO: 43 and the immunoglobulin light chain comprises the amino acid sequence of SEQ ID NO: 44.

53. The polypeptide or polypeptide complex of claim 31, wherein the immunoglobulin light chain and the immunoglobulin heavy chain comprise a set of six CDR sequences of
CDR1-H: SEQ ID NO: 7, CDR2-H: SEQ ID NO: 13, CDR3-H: SEQ ID NO: 19,
CDR1-L: SEQ ID NO: 25, CDR2-L: SEQ ID NO: 31, CDR3-L: SEQ ID NO: 37;
and wherein the immunoglobulin heavy chain comprises an amino acid sequence of at least 95% sequence identity to SEQ ID NO: 75 and the immunoglobulin light chain comprises an amino acid sequence of at least 95% sequence identity to SEQ ID NO: 76.

54. The polypeptide or polypeptide complex of claim 53, wherein the immunoglobulin heavy chain comprises an amino acid sequence of at least 99% sequence identity to SEQ ID NO: 75 and the immunoglobulin light chain comprises an amino acid sequence of at least 99% sequence identity to SEQ ID NO: 76.

55. The polypeptide or polypeptide complex of claim 53, wherein the immunoglobulin heavy chain comprises the amino acid sequence of SEQ ID NO: 75 and the immunoglobulin light chain comprises the amino acid sequence of SEQ ID NO: 76.

56. The polypeptide or polypeptide complex of claim 31, wherein the immunoglobulin light chain and the immunoglobulin heavy chain comprise a set of six CDR sequences of
CDR1-H: SEQ ID NO: 7, CDR2-H: SEQ ID NO: 13, CDR3-H: SEQ ID NO: 19,
CDR1-L: SEQ ID NO: 25, CDR2-L: SEQ ID NO: 31, CDR3-L: SEQ ID NO: 37;
and wherein the immunoglobulin heavy chain comprises an amino acid sequence of at least 95% sequence identity to SEQ ID NO: 45 and the immunoglobulin light chain comprises an amino acid sequence of at least 95% sequence identity to SEQ ID NO: 46.

57. The polypeptide or polypeptide complex of claim 56, wherein the immunoglobulin heavy chain comprises an amino acid sequence of at least 99% sequence identity to SEQ ID NO: 45 and the immunoglobulin light chain comprises an amino acid sequence of at least 99% sequence identity to SEQ ID NO: 46.

58. The polypeptide or polypeptide complex of claim 56, wherein the immunoglobulin heavy chain comprises the amino acid sequence of SEQ ID NO: 45 and the immunoglobulin light chain comprises the amino acid sequence of SEQ ID NO: 46.

59. The polypeptide or polypeptide complex of claim 31, wherein the immunoglobulin light chain and the immunoglobulin heavy chain comprise a set of six CDR sequences of
CDR1-H: SEQ ID NO: 8, CDR2-H: SEQ ID NO: 14, CDR3-H: SEQ ID NO: 20,
CDR1-L: SEQ ID NO: 26, CDR2-L: SEQ ID NO: 32, CDR3-L: SEQ ID NO: 38;
and wherein the immunoglobulin heavy chain comprises an amino acid sequence of at least 95% sequence identity to SEQ ID NO: 77 and the immunoglobulin light chain comprises an amino acid sequence of at least 95% sequence identity to SEQ ID NO: 78.

60. The polypeptide or polypeptide complex of claim 59, wherein the immunoglobulin heavy chain comprises an amino acid sequence of at least 99% sequence identity to SEQ ID NO: 77 and the immunoglobulin light chain comprises an amino acid sequence of at least 99% sequence identity to SEQ ID NO: 78.

61. The polypeptide or polypeptide complex of claim 59, wherein the immunoglobulin heavy chain comprises the amino acid sequence of SEQ ID NO: 77 and the immunoglobulin light chain comprises the amino acid sequence of SEQ ID NO: 78.

62. The polypeptide or polypeptide complex of claim 31, wherein the immunoglobulin light chain and the immunoglobulin heavy chain comprise a set of six CDR sequences of
CDR1-H: SEQ ID NO: 8, CDR2-H: SEQ ID NO: 14, CDR3-H: SEQ ID NO: 20,
CDR1-L: SEQ ID NO: 26, CDR2-L: SEQ ID NO: 32, CDR3-L: SEQ ID NO: 38;
and wherein the immunoglobulin heavy chain comprises an amino acid sequence of at least 95% sequence identity to SEQ ID NO: 47 and the immunoglobulin light chain comprises an amino acid sequence of at least 95% sequence identity to SEQ ID NO: 48.

63. The polypeptide or polypeptide complex of claim 62, wherein the immunoglobulin heavy chain comprises an amino acid sequence of at least 99% sequence identity to SEQ ID NO: 47 and the immunoglobulin light chain comprises an amino acid sequence of at least 99% sequence identity to SEQ ID NO: 48.

64. The polypeptide or polypeptide complex of claim 62, wherein the immunoglobulin heavy chain comprises the amino acid sequence of SEQ ID NO: 47 and the immunoglobulin light chain comprises the amino acid sequence of SEQ ID NO: 48.

65. The polypeptide or polypeptide complex of claim 31, wherein the immunoglobulin heavy chain comprises the amino acid sequence of SEQ ID NO: 79 and the immunoglobulin light chain comprises the amino acid sequence of SEQ ID NO: 80.

66. The polypeptide or polypeptide complex of claim 1, wherein the antibody or antigen binding fragment thereof comprises the amino acid sequence of SEQ ID NO: 89.

67. The polypeptide or polypeptide complex of claim 1, wherein the antibody or antigen binding fragment thereof comprises the amino acid sequence of SEQ ID NO: 90.

68. The polypeptide or polypeptide complex of claim 1, wherein the immunoglobulin heavy chain comprises amino acid mutations K154E and K220E wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 90% sequence identity to SEQ ID NO: 83 and the immunoglobulin light chain comprises an amino acid sequence of at least 90% sequence identity to SEQ ID NO: 84.

69. The polypeptide or polypeptide complex of claim 1, wherein the immunoglobulin heavy chain comprises amino acid mutations K154E and K220E wherein the position of the amino acid mutations corresponds to that of SEQ ID NO: 1 and the immunoglobulin light chain comprises amino acid mutations E129R and Q130K wherein the position of the amino acid mutations corresponds to that of SEQ ID NO: 2, and wherein the immunoglobulin heavy chain comprises an amino acid sequence of at least 95% sequence identity to SEQ ID NO: 83 and the immunoglobulin light chain comprises an amino acid sequence of at least 95% sequence identity to SEQ ID NO: 84.

70. The polypeptide or polypeptide complex of claim 1, wherein the immunoglobulin heavy chain comprises an amino acid sequence of at least 99% sequence identity to SEQ ID NO: 49 and the immunoglobulin light chain comprises an amino acid sequence of at least 99% sequence identity to SEQ ID NO: 50.

71. The polypeptide or polypeptide complex of claim 1, wherein the immunoglobulin heavy chain comprises an amino acid sequence of at least 99% sequence identity to SEQ ID NO: 51 and the immunoglobulin light chain comprises an amino acid sequence of at least 99% sequence identity to SEQ ID NO: 52.

72. The polypeptide or polypeptide complex of claim 1, wherein the immunoglobulin heavy chain comprises the amino acid sequence of SEQ ID NO: 51 and the immunoglobulin light chain comprises the amino acid sequence of SEQ ID NO: 52.

73. The polypeptide or polypeptide complex of claim 1, wherein the immunoglobulin heavy chain comprises an amino acid sequence of at least 99% sequence identity to SEQ ID NO: 53 and the immunoglobulin light chain comprises an amino acid sequence of at least 99% sequence identity to SEQ ID NO: 54.

74. The polypeptide or polypeptide complex of claim 1, wherein the immunoglobulin heavy chain comprises the amino acid sequence of SEQ ID NO: 53 and the immunoglobulin light chain comprises the amino acid sequence of SEQ ID NO: 54.

75. The polypeptide or polypeptide complex of claim 1, wherein the immunoglobulin heavy chain comprises an amino acid sequence of at least 99% sequence identity to SEQ ID NO: 55 and the immunoglobulin light chain comprises an amino acid sequence of at least 99% sequence identity to SEQ ID NO: 56.

76. The polypeptide or polypeptide complex of claim 1, wherein the immunoglobulin heavy chain comprises the amino acid sequence of SEQ ID NO: 55 and the immunoglobulin light chain comprises the amino acid sequence of SEQ ID NO: 56.

77. The polypeptide or polypeptide complex of claim 1, wherein the immunoglobulin heavy chain comprises an amino acid sequence of at least 99% sequence identity to SEQ ID NO: 57 and the immunoglobulin light chain comprises an amino acid sequence of at least 99% sequence identity to SEQ ID NO: 58.

78. The polypeptide or polypeptide complex of claim 1, wherein the immunoglobulin heavy chain comprises the amino acid sequence of SEQ ID NO: 57 and the immunoglobulin light chain comprises the amino acid sequence of SEQ ID NO: 58.

79. The polypeptide or polypeptide complex of claim 1, wherein the immunoglobulin heavy chain comprises an amino acid sequence of at least 99% sequence identity to SEQ ID NO: 59 and the immunoglobulin light chain comprises an amino acid sequence of at least 99% sequence identity to SEQ ID NO: 60.

80. The polypeptide or polypeptide complex of claim 1, wherein the immunoglobulin heavy chain comprises the amino acid sequence of SEQ ID NO: 59 and the immunoglobulin light chain comprises the amino acid sequence of SEQ ID NO: 60.

81. The polypeptide or polypeptide complex of claim 1, wherein the polypeptide or polypeptide complex that comprises the antibody or antigen binding fragment thereof that binds to STEAP-1 is a monoclonal antibody.

82. The polypeptide or polypeptide complex of claim 1, wherein the polypeptide or polypeptide complex that comprises the antibody or antigen binding fragment thereof that binds to STEAP-1 is a monospecific antibody, bispecific antibody, or trispecific antibody.

83. The polypeptide or polypeptide complex of claim 1, wherein the antibody or antigen binding fragment thereof that binds to STEAP-1 comprises a Fab, Fab′, (Fab′)₂ or a single chain variable fragment (scFv).

84. The polypeptide or polypeptide complex of claim 83, wherein the antibody or antigen binding fragment thereof that binds to STEAP-1 comprises the Fab.

85. The polypeptide or polypeptide complex of claim 83, wherein the antibody or antigen binding fragment thereof that binds to STEAP-1 comprises the scFv.

86. The polypeptide or polypeptide complex of claim 85, wherein a C-terminus of the immunoglobulin light chain of the scFv is connected to an N-terminus of the immunoglobulin heavy chain of the scFv via a linker (L).

87. The polypeptide or polypeptide complex of claim 85, wherein a C-terminus of the immunoglobulin heavy chain of the scFv is connected to an N-terminus of the immunoglobulin light chain of the scFv via a linker (L).

88. The polypeptide or polypeptide complex of claim 1, wherein the polypeptide or polypeptide complex comprises more than one antibody or antigen binding fragment thereof that binds to STEAP-1.

89. The polypeptide or polypeptide complex of claim 1, wherein the polypeptide or polypeptide complex comprises more than one antibody or antigen binding fragment thereof that binds to STEAP-1 and the more than one antibody or antigen binding fragment thereof that binds to STEAP-1 comprises two Fab or Fab′, or a Fab and a Fab′, that are connected by a linker (L1).

90. The polypeptide or polypeptide complex of claim 89, wherein L₁ connects an immunoglobulin heavy chain of the Fab or Fab′ that binds STEAP-1 with another immunoglobulin heavy chain of the second Fab or Fab′ that binds STEAP-1.

91. The polypeptide or polypeptide complex of claim 89, wherein L₁ connects an immunoglobulin light chain of the Fab or Fab′ that binds STEAP-1 with another immunoglobulin light chain of the second Fab or Fab′ that binds STEAP-1.

92. The polypeptide or polypeptide complex of claim 89, wherein L₁ connects an immunoglobulin light chain of the Fab or Fab′ that binds STEAP-1 with an immunoglobulin heavy chain of the second Fab or Fab′ that binds STEAP-1.

93. The polypeptide or polypeptide complex of claim 89, wherein L₁ comprises an amino acid sequence selected from any one of SEQ ID NOs: 91-96.

94. The polypeptide or polypeptide complex of claim 1, wherein the polypeptide or polypeptide complex comprises an antibody or antigen binding fragment thereof that binds to a second antigen other than STEAP-1.

95. A pharmaceutical composition comprising:
(i) the polypeptide or polypeptide complex of claim 1; and
(ii) a pharmaceutically acceptable excipient.

96. An isolated recombinant nucleic acid molecule encoding a polypeptide of the antibody or antigen binding fragment thereof of claim 1.

97. An expression vector comprising a nucleic acid encoding a polypeptide of the antibody or antigen binding fragment thereof of claim 1.

98. A host cell comprising a polypeptide of the antibody or antigen binding fragment thereof of claim 1.

99. A method of treating a cancer in a subject in need thereof comprising administering to the subject the polypeptide or polypeptide complex of claim 1.

100. The method of claim 99, wherein the cancer is sarcoma or a cancer of the prostate, breast, pancreas, bladder, gastrointestinal tract, testis, ovary, or cervix.

101. The method of claim 99, wherein the cancer is prostate cancer or Ewing sarcoma.

102. The method of claim 99, further comprising administering to the subject an immune checkpoint inhibitor.

103. The method of claim 102, wherein the immune checkpoint inhibitor comprises an antibody or antigen binding fragment thereof that binds to PD-1.


SEQUENCE LISTING (106 sequences)

SEQ ID NO: 1 - protein, 227 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 2 - protein, 220 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 6 aa, synthetic construct
SDYAWN

SEQ ID NO: 4 - protein, 6 aa, synthetic construct
SDYAWN

SEQ ID NO: 5 - protein, 6 aa, synthetic construct
SDYAMN

SEQ ID NO: 6 - protein, 6 aa, synthetic construct
SDYAMN

SEQ ID NO: 7 - protein, 6 aa, synthetic construct
SDYAMN

SEQ ID NO: 8 - protein, 6 aa, synthetic construct
SDYAMN

SEQ ID NO: 9 - protein, 16 aa, synthetic construct
YISNSGSTSYNPSLKS

SEQ ID NO: 10 - protein, 16 aa, synthetic construct
YISNSGSISYNPSLKS

SEQ ID NO: 11 - protein, 16 aa, synthetic construct
YISNSGSTSYNPSVKS

SEQ ID NO: 12 - protein, 16 aa, synthetic construct
YISNSGSISYNPSVKG

SEQ ID NO: 13 - protein, 16 aa, synthetic construct
YISNSGSTSYNPSVKS

SEQ ID NO: 14 - protein, 16 aa, synthetic construct
YISNSGSTSYNPSVKS

SEQ ID NO: 15 - protein, 15 aa, synthetic construct
ERNYDYDDYYYAMDY

SEQ ID NO: 16 - protein, 15 aa, synthetic construct
ERNYDYDDYYYAMDY

SEQ ID NO: 17 - protein, 15 aa, synthetic construct
ERNYDYDDYYYAMDY

SEQ ID NO: 18 - protein, 15 aa, synthetic construct
ERNYDYDDYYYAMDY

SEQ ID NO: 19 - protein, 15 aa, synthetic construct
DRNYDYDDYYYAFDY

SEQ ID NO: 20 - protein, 15 aa, synthetic construct
DRNYDYDDYYYAFDY

SEQ ID NO: 21 - protein, 17 aa, synthetic construct
KSSQSLLYRSNQKNYLA

SEQ ID NO: 22 - protein, 17 aa, synthetic construct
KSSQSLLYRSNQKNYLA

SEQ ID NO: 23 - protein, 17 aa, synthetic construct
KSSQSLLYRSNNKNYLA

SEQ ID NO: 24 - protein, 17 aa, synthetic construct
KSSQSLLYRSNNKNYLA

SEQ ID NO: 25 - protein, 17 aa, synthetic construct
KSSQSLLYRSNNKNYLA

SEQ ID NO: 26 - protein, 17 aa, synthetic construct
KSSQSLLYRSNNKNYLA

SEQ ID NO: 27 - protein, 7 aa, synthetic construct
WASTRES

SEQ ID NO: 28 - protein, 7 aa, synthetic construct
WASTRES

SEQ ID NO: 29 - protein, 7 aa, synthetic construct
WASTRES

SEQ ID NO: 30 - protein, 7 aa, synthetic construct
WASTRES

SEQ ID NO: 31 - protein, 7 aa, synthetic construct
WASTRES

SEQ ID NO: 32 - protein, 7 aa, synthetic construct
WASTRES

SEQ ID NO: 33 - protein, 9 aa, synthetic construct
QQYYNYPRT

SEQ ID NO: 34 - protein, 9 aa, synthetic construct
QQYYNYPRT

SEQ ID NO: 35 - protein, 9 aa, synthetic construct
QQYYNYPRT

SEQ ID NO: 36 - protein, 9 aa, synthetic construct
QQYYNYPRT

SEQ ID NO: 37 - protein, 9 aa, synthetic construct
QQYYNYPRT

SEQ ID NO: 38 - protein, 9 aa, synthetic construct
QQYYNYPRT

SEQ ID NO: 39 - protein, 227 aa, synthetic construct
EVQLVESGGGLVQPGGSLRLSCAASGFSITSDYAWNWVRQAPGKGLEWVGYISNSGSISYNPSLKSRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 40 - protein, 220 aa, synthetic construct
DIVMTQSPDSLAVSLGERATINCKSSQSLLYRSNQKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 227 aa, synthetic construct
EVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 42 - protein, 220 aa, synthetic construct
DIQMTQSPSSLSASVGDRVTITCKSSQSLLYRSNNKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 227 aa, synthetic construct
EVQLVESGGGLVQPGGSLRLSCAASGFSITSDYAMNWVRQAPGKGLEWVGYISNSGSISYNPSVKGRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 44 - protein, 220 aa, synthetic construct
DIVMTQSPDSLAVSLGERATINCKSSQSLLYRSNNKNYLAWYQQKPGQPPKLLIYWASTRESGVPDRFSGSGSGTDFTLTISSLQAEDVAVYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 227 aa, synthetic construct
EVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 46 - protein, 220 aa, synthetic construct; identical to SEQ ID NO: 42

SEQ ID NO: 47 - protein, 227 aa, synthetic construct
EVQLVESGGGLVQPGGSLRLSCAASGFSITSDYAMNWVRQAPGKGLEWVGYISNSGSTSYNPSVKSRFTISRDNSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 48 - protein, 220 aa, synthetic construct; identical to SEQ ID NO: 44

SEQ ID NO: 49 - protein, 454 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 220 aa, synthetic construct; identical to SEQ ID NO: 2

SEQ ID NO: 51 - protein, 454 aa, synthetic construct
EVQLVESGGGLVQPGGSLRLSCAASGFSITSDYAWNWVRQAPGKGLEWVGYISNSGSISYNPSLKSRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 220 aa, synthetic construct; identical to SEQ ID NO: 40

SEQ ID NO: 53 - protein, 454 aa, synthetic construct
EVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 220 aa, synthetic construct; identical to SEQ ID NO: 42

SEQ ID NO: 55 - protein, 454 aa, synthetic construct
EVQLVESGGGLVQPGGSLRLSCAASGFSITSDYAMNWVRQAPGKGLEWVGYISNSGSISYNPSVKGRFTISRDNSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 220 aa, synthetic construct; identical to SEQ ID NO: 44

SEQ ID NO: 57 - protein, 454 aa, synthetic construct
EVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 220 aa, synthetic construct; identical to SEQ ID NO: 42

SEQ ID NO: 59 - protein, 454 aa, synthetic construct
EVQLVESGGGLVQPGGSLRLSCAASGFSITSDYAMNWVRQAPGKGLEWVGYISNSGSTSYNPSVKSRFTISRDNSKNTLYLQMNSLRAEDTAVYYCARDRNYDYDDYYYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 220 aa, synthetic construct; identical to SEQ ID NO: 44

SEQ ID NO: 61 - protein, 124 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 62 - protein, 113 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 63 - protein, 5 aa, synthetic construct
TYWIE

SEQ ID NO: 64 - protein, 17 aa, synthetic construct
EILPGSGQTDFNEKFQG

SEQ ID NO: 65 - protein, 12 aa, synthetic construct
WGYYGTRGYFNV

SEQ ID NO: 66 - protein, 10 aa, synthetic construct
RASSSVSYMH

SEQ ID NO: 67 - protein, 7 aa, synthetic construct
STSNLAS

SEQ ID NO: 68 - protein, 9 aa, synthetic construct
QQRRSFPYT

SEQ ID NO: 69 - protein, 124 aa, synthetic construct
EVQLVESGGGLVQPGGSLRLSCAASGFSITSDYAWNWVRQAPGKGLEWVGYISNSGSISYNPSLKSRFTISRDNSKNTLYLQMNSLRAEDTAVYYCARERNYDYDDYYYAMDYWGQGTLVTVSS

SEQ ID NO: 70 - protein, 113 aa, synthetic construct
DIVMTQSPDSLAVSLGERATINCKSSQSLLYRSNQKNYLAWYQQKPGQPPKLLIYWASTRESGVPDRFSGSGSGTDFTLTISSLQAEDVAVYYCQQYYNYPRTFGQGTKVEIK

SEQ ID NO: 71 - protein, 124 aa, synthetic construct
EVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARERNYDYDDYYYAMDYWGQGTLVTVSS

SEQ ID NO: 72 - protein, 113 aa, synthetic construct
DIQMTQSPSSLSASVGDRVTITCKSSQSLLYRSNNKNYLAWYQQKPGKAPKLLIYWASTRESGVPSRFSGSGSGTDFTLTISSLQPEDFATYYCQQYYNYPRTFGQGTKVEIK

SEQ ID NO: 73 - protein, 124 aa, synthetic construct
EVQLVESGGGLVQPGGSLRLSCAASGFSITSDYAMNWVRQAPGKGLEWVGYISNSGSISYNPSVKGRFTISRDNSKNTLYLQMNSLRAEDTAVYYCARERNYDYDDYYYAMDYWGQGTLVTVSS

SEQ ID NO: 74 - protein, 113 aa, synthetic construct
DIVMTQSPDSLAVSLGERATINCKSSQSLLYRSNNKNYLAWYQQKPGQPPKLLIYWASTRESGVPDRFSGSGSGTDFTLTISSLQAEDVAVYYCQQYYNYPRTFGQGTKVEIK

SEQ ID NO: 75 - protein, 124 aa, synthetic construct
EVQLVESGGGLVQPGGSLRLSCAVSGYSITSDYAMNWVRQAPGKGLEWVGYISNSGSTSYNPSVKSRFTISRDTSKNTLYLQMNSLRAEDTAVYYCARDRNYDYDDYYYAFDYWGQGTLVTVSS

SEQ ID NO: 76 - protein, 113 aa, synthetic construct; identical to SEQ ID NO: 72

SEQ ID NO: 77 - protein, 124 aa, synthetic construct
EVQLVESGGGLVQPGGSLRLSCAASGFSITSDYAMNWVRQAPGKGLEWVGYISNSGSTSYNPSVKSRFTISRDNSKNTLYLQMNSLRAEDTAVYYCARDRNYDYDDYYYAFDYWGQGTLVTVSS

SEQ ID NO: 78 - protein, 113 aa, synthetic construct; identical to SEQ ID NO: 74

SEQ ID NO: 79 - protein, 124 aa, synthetic construct
EVQLVESGGGLVQPGGSLRLSCAVSGYSITSDYAWNWVRQAPGKCLEWVGYISNSGSTSYNPSLKSRFTISRDTSKNTLYLQMNSLRAEDTAVYYCARERNYDYDDYYYAMDYWGQGTLVTVSS

SEQ ID NO: 80 - protein, 113 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGCGTKVEIK

SEQ ID NO: 81 - protein, 124 aa, synthetic construct; identical to SEQ ID NO: 79

SEQ ID NO: 82 - protein, 113 aa, synthetic construct; identical to SEQ ID NO: 80

SEQ ID NO: 83 - protein, 227 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNGKVDEKVEPKSC

SEQ ID NO: 84 - protein, 220 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 224 aa, synthetic construct
QVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 86 - protein, 213 aa, synthetic construct
EIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 252 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 88 - protein, 252 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKGGGGSGGGGSGGGGSEVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 89 - protein, 252 aa, synthetic construct
EVQLVESGGGLVQPGGSLRLSCAVSGYSITSDYAWNWVRQAPGKCLEWVGYISNSGSTSYNPSLKSRFTISRDTSKNTLYLQMNSLRAEDTAVYYCARERNYDYDDYYYAMDYWGQGTLVTVSSGGGGSGGGGSGGGGSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGCGTKVEIK

SEQ ID NO: 90 - protein, 252 aa, synthetic construct
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGCGTKVEIKGGGGSGGGGSGGGGSEVQLVESGGGLVQPGGSLRLSCAVSGYSITSDYAWNWVRQAPGKCLEWVGYISNSGSTSYNPSLKSRFTISRDTSKNTLYLQMNSLRAEDTAVYYCARERNYDYDDYYYAMDYWGQGTLVTVSS

SEQ ID NO: 91 - protein, 15 aa, synthetic construct
GGGGSGGGGSGGGGS

SEQ ID NO: 92 - protein, 5 aa, synthetic construct
GGGGS

SEQ ID NO: 93 - protein, 9 aa, synthetic construct
GGGGSGGGS

SEQ ID NO: 94 - protein, 10 aa, synthetic construct
GGGGSGGGGS

SEQ ID NO: 95 - protein, 20 aa, synthetic construct
GGGGSGGGGSGGGGSGGGGS

SEQ ID NO: 96 - protein, 25 aa, synthetic construct
GGGGSGGGGSGGGGSGGGGSGGGGS

SEQ ID NO: 97 - protein, 124 aa, synthetic construct; identical to SEQ ID NO: 61

SEQ ID NO: 98 - protein, 113 aa, synthetic construct; identical to SEQ ID NO: 62

SEQ ID NO: 99 - protein, 121 aa, synthetic construct [sequence omitted]

SEQ ID NO: 100 - protein, 106 aa, synthetic construct [sequence omitted]

SEQ ID NO: 101 - protein, 124 aa, synthetic construct; identical to SEQ ID NO: 61

SEQ ID NO: 102 - protein, 113 aa, synthetic construct; identical to SEQ ID NO: 62

SEQ ID NO: 103 - protein, 124 aa, synthetic construct; identical to SEQ ID NO: 61

SEQ ID NO: 104 - protein, 113 aa, synthetic construct; identical to SEQ ID NO: 62

SEQ ID NO: 105 - protein, 242 aa, synthetic construct
QVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSSGGGGSGGGGSGGGGSEIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIK

SEQ ID NO: 106 - protein, 242 aa, synthetic construct
EIVLTQSPATLSLSPGERATLSCRASSSVSYMHWFQQKPGQAPRLLIYSTSNLASGIPARFSGSGSGTDYTLTISSLEPEDFAVYYCQQRRSFPYTFGQGTKLEIKGGGGSGGGGSGGGGSQVQLVQSGAEVKKPGASVKVSCKASGYTFSTYWIEWVRQAPGQRLEWMGEILPGSGQTDFNEKFQGRVTFTADTSSDTAYMELSSLRSEDTAVYYCTRWGYYGTRGYFNVWGQGTLVTVSS